(19) United States
(12) Patent Application Publication
(10) Pub. No.: US 2022/0118253 A1
(43) Pub. Date: Apr. 21, 2022

(54) SYSTEMS AND METHODS FOR CRANIOCERVICAL AND AURICULAR NEUROMODULATION

(71) Applicant: IST, LLC, Dover, DE (US)

(72) Inventors: Daniel Z. WETMORE, Brooklyn, NY (US); William J. TYLER, Cave Creek, AZ (US)

(21) Appl. No.: 17/423,747

(22) PCT Filed: Jan. 21, 2020
(86) PCT No.: PCT/US2020/014430

§ 371 Date: Jul. 16, 2021

Related U.S. Application Data
(60) Provisional application No. 62/794,549, filed on Jan. 18, 2019.

(51) Int. Cl. A61N 1/36; A61N 1/04; A61N 1/05; H04R 1/10

(52) U.S. Cl. CPC A61N 1/36034; A61N 1/0456; H04R 1/1016; A61N 1/0526; A61N 1/0472

(57) ABSTRACT

Methods and apparatuses for non-invasive auricular and/or cervical stimulation to modulate multiple integrated neural networks. For example described herein are methods and apparatuses (e.g., devices, systems, etc.) for modulating nerves located at the base of the skull or in the neck, such as craniocervical nerves, cranial nerves, or cervical spinal nerves and/or the auricular nerve for influencing one or more biological homeostasis and related physiological processes.

### CROSS REFERENCE TO RELATED APPLICATIONS

This patent application claims priority to U.S. provisional patent application No. 62/794,549, titled “SYSTEMS AND METHODS FOR CRANIOCERVICAL AND AURICULAR NEUROMODULATION,” and filed on Jan. 18, 2019, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Apparatuses and methods for applying non-invasive auricular and cervical stimulation to modulate multiple integrated neural networks.

### BACKGROUND

Modulation of neural circuits to adjust biological homeostasis could allow the modification of numerous potentially useful physiological processes, including sleep, appetite, body weight, and body temperature. In particular, it would be beneficial to non-invasively modulate these processes.

In addition, the ability to altering the activity of neural circuits including the craniocervical, cranial, or cervical nerves and/or auricular nerves may allow the improvement of mental capacity for problem solving or learning. These circuits may also be used to enhance attention by broadening or focusing it or by increasing working memory, including potentially by altering activity during consolidation phases of human information storage such as during sleep or specific phases of sleep.

Effective and reliable control of such processes has proven difficult to achieve. Described herein are methods and apparatuses that may allow such modulation.

### SUMMARY OF THE DISCLOSURE

In general, described herein are methods and apparatuses for non-invasive auricular and cervical stimulation to modulate multiple integrated neural networks. For example described herein are methods and apparatuses (e.g., devices, systems, etc.) for modulating nerves located at the base of the skull or in the neck, such as craniocervical nerves, cranial nerves, or cervical spinal nerves and/or the auricular nerve for influencing one or more biological homeostasis and related physiological processes. Also described are methods and apparatuses for modulating auricular nerves for influencing biological homeostasis and related physiological processes. The modulation methods and apparatuses that influence biological homeostasis and related physiological processes may have effects on various processes including sleep, appetite, body weight, and body temperature. For example, these methods and apparatus may be used to selectively and specifically trigger one or more behaviors such as sleep or feeding or to terminate such behaviors by inducing waking or satiety, respectively. Further, the methods and apparatuses described herein may modulate the activity of cranial and cervical nerves and/or auricular nerves to stimulate metabolism or alter activity of metabolic processes to decrease body weight or inhibit weight gain or induce weight loss through other mechanisms such as regulating body temperature or act through a combination of processes.

Thus, described herein are methods and devices for altering the activity of craniocervical, cranial, or cervical nerves and/or auricular nerves to alter, modify, and/or improve mental capacity for problem solving or learning. Such methods and apparatuses may be used to enhance attention by broadening or focusing it or by increasing working memory, but may also work by altering activity during consolidation phases of human information storage such as during sleep or specific phases of sleep.

In some variations, described herein are apparatuses for performing any of the methods described herein. For example, an apparatus may generally include one or more earpieces that are configured to be worn in the ear canal and/or the outer portion of the ear (e.g., the pinna) and include one or more contact regions for selectively applying a stimulation signal, such as an electrical stimulation signal and/or a thermal stimulation signal and/or a mechanical stimulation signal and/or an optical stimulation signal; the apparatus may also include a neck piece (e.g., neck contact) configured to be worn on the neck (e.g., the back of the neck) including one or more contact region configured to apply a neck simulation signal (e.g., an electrical, thermal, mechanical, optical, etc. stimulation signal); and a controller configured to concurrently apply both the stimulation signal(s) from the earpiece and the stimulation signal(s) from the neck piece (neck contact). Either or both the earpiece(s) and the neckpiece may include a hydrogel forming all or part of the contact including the contact regions configured to apply the stimulation signals. The controller may be connected to the earpiece(s) and/or the neckpieces and may be wired or wireless connect, or integrated into one or the other (or both) of the neckpieces and/or earpieces. In some variations the earpiece may include multiple contact regions. The multiple contact regions may be arranged on a projection into the ear canal and/or on other regions configured to contact the outer ear. The neck piece may include one or more contacts.

In some variations the stimulation signals are electric. For example, an apparatus may include a first low-impedance and compressible hydrogel configured to fit into a first ear canal so that the hydrogel expands to contact a wall of the first ear canal, wherein the first hydrogel comprises a first electrical contact; a second low-impedance and compressible hydrogel configured to fit into a second ear canal so that the hydrogel expands to contact a wall of the second ear canal, wherein the second hydrogel comprises a second electrical contact; a neck contact (e.g., neck piece) configured to be worn on the back of a subject's neck, the neck contact comprising one or more neck electrical contacts; and a controller coupled with the first and second electrical contacts and the one or more neck electrical contacts, the controller configured to deliver a first treatment electrical signal between the first and second electrical contacts and a second treatment electrical signal to the one or more neck contacts, wherein the first treatment electrical signal comprises a biphasic, pulsed signal having a frequency of greater than 200 Hz to deliver a current density of greater than 2 mA/cm².

The controller may be configured to concurrently deliver the first and second treatment electrical signals. The second treatment electrical signal may be different than the first treatment electrical signal, and may comprise a biphasic, pulsed signal having a frequency of greater than 200 Hz. The second treatment signal may comprises a biphasic, pulsed signal having a frequency of greater than 200 Hz to deliver a current density of greater than 2 mA/cm². The first and second hydrogel may each have an impedance of less than 1.5 KOhms (e.g., 1.2 KOhms or less, 1 KOhm or less, 0.9 KOhms or less, 0.8 KOhms or less, 0.7 KOhms or less, between 100-600 Ohms, between 200-500 Ohms, etc.). The hydrogel may comprises a silicone hydrogel. In some variations the hydrogel may have a water content that is between 40-95% (e.g., between 40-50%, between 40-60%, between 50-60%, between 50-75%, between 60-70%, between 60-85%, between 70-80%, between 70-90%, between 80-95%, etc.). In some variations the hydrogel is a double network hydrogel. In some variations, the first and second hydrogel comprises a silicone hydrogel.

The controller maybe configured to deliver the first and second treatment electrical signals for a predetermined time period, e.g., between about 1 minute and 1 hour, between about 2 minutes and 1 hour, between about 2 minutes and 45 minutes, between about 2 minutes and 30 minutes, between about 2 minutes and 20 minutes, between about 5 minutes and 30 minutes, etc.).

The controller may be housed in a housing. In some variations the controller may include one more display screens, one or more memories, one or more processors, one or more power sources (e.g., batteries, capacitors, etc.), and/or one or more controls (e.g., buttons). The controller may be configured to display information for subject feedback, such as sensed subject-specific information (e.g., temperature, blood pressure, heart rate, etc.). The controller may display timing information (including time to next dose, duration of dose, etc.).

The controller may be configured to deliver the treatment signal (e.g., treatment electrical signal) for a predetermined time period of between about 0.2 minutes and 30 minutes (e.g., between about 0.5 min and 30 minutes, between about 1 min and 30 minutes, between about 5 minutes and 30 minutes, etc.).

The hydrogel may be divided up into different (and independent) contact regions. For example, the first hydrogel may include the first electrical contact and a third electrical contact that may be insulated from each other; the second hydrogel may include the second electrical contact and a fourth electrical contact. As mentioned, the controller may be configured as a multimodal controller configured to separately control the application of electrical signals between the first, second, third and fourth electrical contacts (or additional contacts). Thus, the first electrical contact may be electrically isolated from the third electrical contact.

Any of the apparatuses described herein may include a speaker configured to emit audio signals into the first ear canal when the hydrogel is worn in the first ear canal. Thus, the first (and/or second) hydrogel may be hollow, having a central region through which sound may be transmitted. In some variations the controller may be configured to drive the speaker to emit an audio signal (e.g., a tone, sequence of tones, chords, etc.) in conjunction with the treatment electrical signal.

The controller may be configured so that the frequency of the applied electrical signal is greater than about 250 Hz (e.g., 250 Hz or greater, 300 Hz or greater, 350 Hz or greater, 400 Hz or greater, 500 Hz or greater, etc.). The controller may be configured so that the current density that is greater than 2 mA/cm² (e.g., 3 mA/cm² or greater, 4 mA/cm² or greater, 5 mA/cm² or greater, 7 mA/cm² or greater, etc.). The ability of the apparatuses described herein to achieve these relatively high current densities without inducing pain may be due, in part, to the hydrogel, and in particularly to the compression of the highly conductive hydrogel within the ear canal. In any of these apparatuses, the hydrogel may be compressed by 2% or more, 5% or more, 7% or more, 8% or more, 9% or more, 10% or more, 15% or more, etc.

In any of these variations, the controller may be configured as a multimodal controller configured to separately control the application of first and second treatment electrical signals.

In general, the controller may be configured so that the treatment electrical signal has a pulse width of 150 microseconds or less (e.g., 140 microseconds or less, 130 microseconds or less, 120 microseconds or less, 110 microseconds or less, 100 microseconds or less, 80 microseconds or less, 50 microseconds or less, etc.).

Any of the apparatuses described herein may have a hydrogel having a shore A durometer of 80 or less (e.g., between 20-80, between 30-75, between 25-65, between 25-80, 40 or less, 45 or less, 50 or less, 55 or less, 60 or less, 65 or less, 70 or less, 75 or less, etc.).

In some variation the multiple contacts (e.g., electrical contacts, thermal contacts, optical contacts, mechanical contacts, etc.) may be arranged radially and/or along the length of the ear canal when the apparatus is worn. For example, the plurality of electrical contacts may be arranged radially around the first ear canal when the device is worn in the first ear canal.

One or more additional contacts (e.g., electrical, thermal, optical, mechanical, etc.) may be present on the apparatus and configured to contact other regions of the ear (e.g., the pinna, etc.) when the apparatus is worn. For example, the apparatus may include one or more external electrical contacts may be configured to couple with an outer region of an ear containing the first ear canal, wherein the multi-channel controller is further configured to concurrently deliver additional treatment electrical signals to the one or more external electrical contacts while delivering treatment electrical signals between one or more of the first plurality of electrical contacts and one or more of the second plurality of electrical contacts.

Any of the earpieces may include one or more contacts. For example, an apparatus may include: a first low-impedance and compressible hydrogel configured to fit into a first ear canal so that the hydrogel expands to contact a wall of the first ear canal, wherein the first hydrogel comprises a first plurality of electrical contacts; a neck contact configured to be worn on the back of a subject's neck, the neck contact comprising one or more neck electrical contacts; and a controller coupled with the first plurality of electrical contacts and the one or more neck electrical contact, the controller configured as a multi-channel controller configured to independently deliver treatment electrical signals from one or more of the first plurality of electrical contacts and from the one or more neck electrical contacts, wherein the treatment electrical signals each comprise a biphasic, pulsed signal having a frequency of greater than 200 Hz to deliver a current density of greater than 2 mA/cm².

The apparatus may include a second low-impedance and compressible hydrogel configured to fit into a second ear canal so that the hydrogel expands to contact a wall of the second ear canal, wherein the second hydrogel comprises a second plurality of electrical contacts.

The first plurality of electrical contacts may be arranged radially around the first ear canal when the device is worn in the first ear canal, and/or along the length of the ear canal.

As mentioned, the hydrogels described herein may have a low impedance (e.g., each may have an impedance of less than 1.5 KOhms, 1.2 KOhms or less, less than 1 KOhm, less than 900 Ohms, less than 800 Ohms, between 100-600 Ohms, between 200-500 Ohms, etc.).

The hydrogel may be a silicone hydrogel or a hydrogel including silicone. In some variations the hydrogel may have a water content that is between 40-95% (e.g., between 40-50%, between 40-60%, between 50-60%, between 50-75%, between 60-70%, between 60-85%, between 70-80%, between 70-90%, between 80-95%, etc.). In some variations the hydrogel may be a double network hydrogel.

They hydrogel may incorporate one or more of: and antifungal, an antibacterial, deodorant, etc. For example, the hydrogel may contain an additive such as an antibiotic and/or an anti-fungal, and/or other organic compounds to prevent infection and to control odor. For example, the hydrogel may include an anti-microbial agent, such as one or more of: chlorhexidene acetate, chlorhexideine gluconate, chlorhexidine hydrochloride, and chlorhexidine sulfate, silver acetate, silver benzoate, silvercarbonate, silver iodate, silver iodide, silver lactate, silver laurate, silver nitrate, silver oxide, silver palmitate, silver protein, and silver sulfadiazine, polymyxin, tetracycline, tobramycin, gentamicin, rifampician, bacitracin, neomycin, chloramphenical, oxolinic acid, norfloxacin, nalidix acid, pefloxacin, enoxacin, ciprofloxacin, ampicillin, amoxicillin, piracil, cephalosporins, vancomycin, and bismuth tribromophenate. In some variations, the hydrogel may include an anti-fungal agent such as one or more of: Tolnaftate, Miconazole, Fluconazole, Econazole, Ketoconazole, Itraconazole, Terbinafine, Amphotericin, Nystatin and Natamycin. In some variations, the hydrogel may include an anti-odorant such as one or more of: grapefruit Seed Extract, Tea Tree Oil, Myrtle Oil, and Lemon grass extract.

In any of these apparatuses, the hydrogel may include a color (e.g., dye) to indicate the location of the one or more contacts (e.g., electrical contacts, thermal contacts, etc.). The hydrogel may include a stiffener in order to provide additional strength or support when removing/applying (e.g., replacing) the hydrogel on/off of the earpiece, as described above.

The controller may be configured to deliver the treatment signal (e.g., treatment electrical signal) for a predetermined time period of between about 0.2 minutes and 30 minutes (e.g., between about 0.5 min and 30 minutes, between about 1 min and 30 minutes, between about 5 minutes and 30 minutes, etc.).

The hydrogel may be divided up into different (and independent) contact regions. For example, the first hydrogel may include the first electrical contact and a third electrical contact that may be insulated from each other; the second hydrogel may include the second electrical contact and a fourth electrical contact. As mentioned, the controller may be configured as a multimodal controller configured to separately control the application of electrical signals between the first, second, third and fourth electrical contacts (or additional contacts). Thus, the first electrical contact may be electrically isolated from the third electrical contact.

Any of the apparatuses described herein may include a speaker configured to emit audio signals into the first ear canal when the hydrogel is worn in the first ear canal. Thus, the first (and/or second) hydrogel may be hollow, having a central region through which sound may be transmitted. In some variations the controller may be configured to drive the speaker to emit an audio signal (e.g., a tone, sequence of tones, chords, etc.) in conjunction with the treatment electrical signal.

The controller may be configured so that the frequency of the applied electrical signal is greater than about 250 Hz (e.g., 250 Hz or greater, 300 Hz or greater, 350 Hz or greater, 400 Hz or greater, 500 Hz or greater, etc.). The controller may be configured so that the current density that is greater than 2 mA/cm² (e.g., 3 mA/cm² or greater, 4 mA/cm² or greater, 5 mA/cm² or greater, 7 mA/cm² or greater, etc.). The ability of the apparatuses described herein to achieve these relatively high current densities without inducing pain may be due, in part, to the hydrogel, and in particularly to the compression of the highly conductive hydrogel within the ear canal. In any of these apparatuses, the hydrogel may be compressed by 2% or more, 5% or more, 7% or more, 8% or more, 9% or more, 10% or more, 15% or more, etc.

In general, the controller may be configured so that the treatment electrical signal has a pulse width of 150 microseconds or less (e.g., 140 microseconds or less, 130 microseconds or less, 120 microseconds or less, 110 microseconds or less, 100 microseconds or less, 80 microseconds or less, 50 microseconds or less, etc.).

Any of the apparatuses described herein may have a hydrogel having a shore A durometer of 80 or less (e.g., between 20-80, between 30-75, between 25-65, between 25-80, 40 or less, 45 or less, 50 or less, 55 or less, 60 or less, 65 or less, 70 or less, 75 or less, etc.).

In some variation the multiple contacts (e.g., electrical contacts, thermal contacts, optical contacts, mechanical contacts, etc.) may be arranged radially and/or along the length of the ear canal when the apparatus is worn. For example, the plurality of electrical contacts may be arranged radially around the first ear canal when the device is worn in the first ear canal.

One or more additional contacts (e.g., electrical, thermal, optical, mechanical, etc.) may be present on the apparatus and configured to contact other regions of the ear (e.g., the pinna, etc.) when the apparatus is worn. For example, the apparatus may include one or more external electrical contacts may be configured to couple with an outer region of an ear containing the first ear canal, wherein the multi-channel controller is further configured to concurrently deliver additional treatment electrical signals to the one or more external electrical contacts while delivering treatment electrical signals between one or more of the first plurality of electrical contacts and one or more of the second plurality of electrical contacts.

Also described herein are apparatuses configured to deliver thermal signals (e.g., thermal sensory biasing signals) to the subject, e.g., the subject's ear canal. In some variations the apparatus may include multiple contacts for contacting specific regions of the ear canal. For example, the apparatus may include a low-impedance and compressible hydrogel configured to fit into a first ear canal so that the hydrogel expands to contact a wall of the first ear canal, wherein the first hydrogel comprises a first plurality of thermal contacts; a plurality of thermal channels, wherein each thermal contact of the plurality of thermal contacts is coupled to one or more heating/cooling sources by a thermal channel of the plurality of thermal channels; and a controller coupled with the one or more heating/cooling sources, wherein the controller is configured as a multi-channel controller configured to independently deliver thermal treatment to each of the plurality of thermal contacts, wherein the thermal treatment comprises a temperature change of between 0.1 and 5 degrees C.

In some variations the apparatus includes additional modes (e.g., electrical, optical, mechanical), including additional contacts on/within the hydrogel or separate from the hydrogel. For example, an apparatus may include: a low-impedance and compressible hydrogel configured to fit into a first ear canal so that the hydrogel expands to contact a wall of the first ear canal, wherein the first hydrogel comprises a thermal contact and an electrical contact; a thermal channel coupling the thermal contact to a heating/cooling source; and a multi-channel controller configured to independently deliver a treatment electrical signal to the electrical contact and a treatment thermal signal to the thermal contact, wherein the treatment electrical signal comprises a biphasic, pulsed signal having a frequency of greater than 200 Hz to deliver a current density of greater than 2 mA/cm2 and wherein the treatment thermal signal comprises a temperature change of between 0.1 and 5 degrees C.

The one or more heating/cooling sources may be any appropriate heating and/or cooling source. For example, the heating/cooling source may be a thermoelectric cooler (TEC). In some variations the heating/cooling source may be a resistive heater. A heating/cooling source may be an evaporation-based cooling, a fan cooler, etc. The signal used to drive the heating/cooling may be similar to that used to drive the electrical signal (e.g., in frequency, duration, pulse width, etc.) or may be different. For example, the controller may be configured to apply a drive signal comprising a pulsed signal having a frequency of greater than 200 Hz to the one or more heating/cooling sources.

In some variations the plurality of contacts (e.g., thermal contacts, electrical contacts, etc.) are arranged radially around the first ear canal when the device is worn in the first ear canal. Alternatively or additionally, the plurality of contacts may be arranged down the length of a projection (e.g., post, shaft, etc.) extending into the ear canal over which the hydrogel may be positioned. The projection may be hollow and/or may house a speaker, one or more sensors, etc. Any of the apparatuses described herein may include a projection configured to fit into the ear canal over which the hydrogel may be coupled.

The apparatus may include one or more external contacts (e.g. thermal contacts, electrical contacts, etc.) configured to couple with an outer region of an ear when the apparatus is worn in the ear canal, wherein the multi-channel controller is further configured to concurrently deliver additional thermal treatment to the one or more external contacts while delivering treatment (e.g., thermal treatment, electrical treatment, etc.) to the first plurality of contacts.

As mentioned above, the hydrogel may comprises a silicone hydrogel. The hydrogel may have a high electrical conductivity and/or may have a high thermal conductivity. For example, the hydrogel may be both highly electrically conductive and may have a high thermal conductivity.

In general, one or more contacts or wires (e.g., thermal channels) may be configured to couple the thermally contacts of the hydrogel with the heater/cooler and the controller. For example, the apparatus may include one or more thermally conductive wires. Similar to the electrically conductive regions, the projection (e.g., post) may include a contact or plate that couples through the hydrogel with the thermal contacts.

The apparatuses described herein may include one or more thermal sensors configured to determine a temperature within the ear canal. The neckpiece may also or alternatively include one or more thermal sensors. The controller may receive input from the thermal sensors and may use the sensed signals to set or modify the applied signals. Additional sensors (skin conductance, heart rate, blood pressure, etc.) may be included.

In general, the temperature applied by the thermal contact to the skin (e.g., within the ear canal) may be between about 0.1 degrees C. and 5 degrees C. (e.g., between 0.1 degrees C. and 4 degrees C., between 0.5 degrees C. and 5 degrees C., between about 0.1 degrees C. and 3 degrees C., etc.). This may include cooling and/or heating. The temperature difference may be relative to the temperature of the inner ear (body temperature). In some variations the controller is configured to deliver the thermal treatment for a predetermined time period, e.g., between about 0.2 minutes and 30 minutes.

As mentioned, any of these apparatuses may include a speaker configured to emit audio signals into the first ear canal when the first hydrogel is worn in the first ear canal. The controller may be configured to drive the speaker to emit an audio signal in conjunction with the thermal treatment.

Also described herein are methods of applying stimulation to both the subject's ear(s) and the subject's cervical region (e.g., neck, including the back of the neck). Treatment may include prevention (e.g., preventative treatments) and/or curative treatments. The subject may be a human or animal (e.g., mammal).

For example, described herein are methods of modulating activity of a subject's cranial and cervical nerves and/or auricular nerve, the method comprising: applying a first treatment signal from a first contact comprising a first hydrogel within a subject's first ear canal, wherein the hydrogel is expanded to contact a wall of the first ear canal; and applying a second treatment signal from a neck contact on a back of the subject's neck, wherein the first and second treatment signals are concurrently applied, further wherein the first and second treatment signals each comprise either a biphasic, pulsed electrical signal having a frequency of greater than 200 Hz and a current density of >2 mA/cm², or a thermal signal at a frequency of greater than 300 Hz configured to generate a change in temperature of between 0.1 and 3 degrees C.

For example, in some variations the first and second treatment signals may both be pulsed electrical signals, the first and second treatment signals may both be thermal signals, the first treatment signal may be a thermal signal and the second treatment signal may be a pulsed electrical signal, or the first treatment signal may be a pulsed electrical signal and the second treatment signal may be a thermal signal.

In some variations the methods described herein are methods of modulating appetite, body weight, and/or body temperature. The method may be a method of modulating the activity of the subject's cranial and cervical nerves and/or auricular nerves to stimulate metabolism or alter activity of metabolic processes. In some variations, the method is method of improving metal capacity for problem solving, learning and/or enhancing attention.

The first and second treatment signals may be different. For example, the first and second treatment signals may be pulsed electrical treatment signals (e.g., treatment electrical signals) and these electrical signals may be different.

In some variations, applying the first and second treatment signals may include applying at or below a sensory threshold for feeling the applied first and second treatment signals. The first and second treatment signals may be applied for 10 minutes or more. As mentioned, any of these methods may include delivering an audio signal to the subject concurrently with the delivery of the first and second treatment signals.

Applying the first treatment signal may include applying the first treatment signal between the first low impedance hydrogel and a second low-impedance hydrogel in the subject's second ear canal. The frequency of the first treatment signal may be, e.g., greater than 250 Hz (e.g., 300 Hz or greater, 400 Hz or greater, 500 Hz or greater, between 200 and 1000 Hz, between 300 and 900 Hz, between 350 and 850 Hz, etc.).

In any of the methods (and apparatuses) described herein one or more of the contacts (e.g., thermal contacts, electrical contacts, etc.) on the ear piece may be positioned, or configured to be positioned, fairly deep within the ear canal. For example, the contact may be configured to be positioned at least 5 mm from the opening into the ear canal (e.g., >5 mm from the opening, 7.5 mm or greater from the opening, 10 mm or greater from the opening, 12.5 mm or greater from the opening, 15 mm or greater from the opening, etc.). In some variations the contacts are configured to be positioned (when the apparatus is worn and/or the method performed) with the contacts at least halfway (e.g., 50% or more) the distance from the opening of the ear canal to the bend in the ear canal (from the tragus to the first bend), such as 55% or more, 60% or more, 65% or more of the distance to the first bend.

Any of the methods described herein, in addition to applying signals (thermal and/or electrical and/or mechanical and/or light) to the ear canal and neck, may apply concurrently apply stimulation to other regions of the body, including in particular the outer portion of the ear (e.g., the pinna or auricle). For example, any of these methods may include concurrently applying electrical or thermal stimulation to one or more regions of the auricle of the subject's ear.

As used herein a subject may refer to a patient. The subject may be any subject that may benefit from the methods described herein. A subject may be diagnosed or undiagnosed with a condition to be treated using the methods and apparatuses described herein. The subject may be human or non-human (e.g., mammalian).

Also described herein are methods of treating a subject by applying electrical stimulation within one or both of the subject's ear canal and concurrently to the back of the subject's neck. For example, a method of treatment may include: inserting a first low-impedance hydrogel into a subject's first ear canal so that the hydrogel expands to contact a wall of the first ear canal, wherein the first hydrogel comprises a first electrical contact; attaching a neck contact onto the back of the subject's neck; and applying a first treatment electrical signal to the first electrical contact, wherein the treatment electrical signal comprises a biphasic, pulsed signal having a frequency of greater than 200 Hz, so that the wall of the first ear canal receives a current density of greater than 2 mA/cm²; and applying a second treatment electrical signal to the neck contact, wherein the first and second treatment electrical signals are concurrently applied.

In general, the first and second treatment electrical signals may be different. In some variations they may be similar (e.g., similar parameter limits or ranges), but may have different time courses. For example, the second treatment electrical signal may also have a frequency of greater than 200 Hz and a current density of greater than 2 mA/cm². In some variations the thermal treatment signals may be driven by similar parameters to the electrical treatment signals.

As mentioned, the signals described herein within one or both ear canal and cervical region (e.g., neck, back of the neck, etc.) may be applied concurrently. The signals may be identical or similar, or may be different, including different modalities (e.g., thermal, electrical, etc.). As used herein concurrently applied signals may overlap in time. The concurrently applied signals may overlap completely (e.g., may start and stop at the same time), or the second (or additional) signals may start and stop during the duration of first signal, while the first signal is ongoing. In some variations the concurrently applied signals may overlap partially, e.g., the first signal may start before the second signal and may end before or at the same time as the second signal (e.g., may be concurrently overlapping).

In general, the signals (e.g., treatments) applied may be below the threshold for conscious detection by the subject. For example, the applied thermal and/or electrical and/o r mechanical signals applied may be imperceptible or barely perceptible. In some variations, applying the treatment comprises applying at a current density that is at or below a sensory threshold for feeling the applied treatment electrical signal.

As mentioned, these methods may generally be directed to treatments for modifying biological homeostasis and related physiological processes. For example, any of these methods may be methods for modifying sleep, appetite, body weight, and/or body temperature. In some variations the methods are methods of improving mental capacity for problem solving or learning. For example, the methods may be methods of enhancing attention.

The treatments described herein may be applied for any appropriate duration. For example, the signals (electrical, thermal, etc.) may be applied for between 1 second and 10 minutes (e.g., between 1 second and 7 minutes, between 10 seconds and 5 minutes, etc.). The treatments may be applied on demand (e.g., selected by the subject) or on a pre-set schedule, or both. For example, a pre-set schedule may include repeating the simulation every x minutes or hours where x is between 5 minutes and 48 hours (e.g., between 10 minutes and 24 hours, between 15 minutes and 20 hours, between 20 minutes and 14 hours, etc.). The apparatus may be configured to indicate to the user that a dose is due to be applied and/or is being applied. Alternatively or in addition, a treatment may be applied when selected by the user.

In some variation a dose may include repeated applications of the same or (more preferably) different signals. For example, a dose may include applying a first signal (electrical, thermal, etc.) to the first contact (e.g., in the subject's ear canal) concurrent with the application of a second signal (or additional signals) to the second or more contacts (e.g., on the subject's neck, other ear canal, outer ear, etc.), then immediately or after a delay period (e.g., between 0.5 seconds and 15 minutes) applying a third signal (electrical, thermal, etc.) to the first contact, concurrently with the application of a fourth signal (or additional signals) to the second or more contacts. The third signal may be different from the first signal and the fourth signal may be different from the second signal.

As mentioned above, any these methods may include the addition of an audio signal during the treatment.

In some variations the application of one or more (e.g., the first) treatment signals may include applying the treatment signals between the subject's ear canals (e.g., between a first earpiece within the subject's ear canal, and a second earpiece within the subject's right ear canal).

A method of modulating activity of a subject's cranial and cervical nerves and/or auricular nerve may include: applying a first treatment electrical signal from a first electrical contact comprising a first low-impedance hydrogel within a subject's first ear canal, wherein the hydrogel is expanded to contact a wall of the first ear canal; and applying a second treatment electrical signal from a neck electrical contact on a back of the subject's neck, wherein the first treatment electrical signal comprises a biphasic, pulsed signal having a frequency of greater than 200 Hz and a current density of >2 mA/cm², further wherein the first and second treatment electrical signals are concurrently applied. For example, the first and second treatment electrical signals may be repeated with a frequency of less than 24 hours (e.g., less than 20 hours, less than 18 hours, less than 14 hours, less than 12 hours, less than 10 hours, less than 8 hours, etc.).

As mentioned above, applying the first and second treatments may comprise applying at a current density that is at or below a sensory threshold for feeling the applied first and second treatment electrical signals. The first and second treatment electrical signals may be different.

In some variations a method of treatment may include: applying a first thermal or electrical treatment signal to a back of a subject's neck; and applying a second treatment signal from a thermal delivery region of a compressible and thermally-conductive hydrogel within the subject's ear canal, wherein the hydrogel is expanded and contacts a wall of the ear canal; and wherein applying the second treatment signal comprises modulating the temperature of the thermal delivery region with temperature profile having a frequency of greater than 300 Hz that generates a change in temperature of between 0.1 and 3 degrees C., further wherein the first and second treatment signals are concurrently applied. These methods may also include applying a third treatment signal from a second compressible and thermally-conductive hydrogel into the subject's second ear canal by selectively modulating the temperature of a second thermal delivery region of the second hydrogel. The first and second thermal delivery regions may be concurrently modulated using the same temperature profiles or using different temperature profiles.

Any of these methods may include sensing the temperature within the ear canal, and may further include adjusting the temperature profiles based on the sensed temperature.

In general, selectively modulating the temperature may comprises cooling and/or heating. Applying a first thermal or electrical treatment signal to a back of a subject's neck may include applying an electrical treatment signal comprising a biphasic, pulsed signal having a frequency of greater than 200 Hz and a current density of >2 mA/cm². In some variations applying the first thermal or electrical treatment signal to a back of a subject's neck comprises modulating the temperature of a thermal delivery region of an applicator on the back of the subject's neck with temperature profile having a frequency of greater than 300 Hz that generates a change in temperature of between 0.1 and 3 degrees C.

The selective modulation of the temperature of each thermal delivery region may be triggered based on the temperature of the ear canal.

Also described and included herein are apparatuses that are configured (including hardware, software and/or firmware) for performing any of the methods described herein. For example, an apparatus may include: a compressible hydrogel configured to fit into a first ear canal so that the hydrogel expands to contact a wall of the first ear canal, wherein the first hydrogel comprises a first thermal contact; a neck thermal contact configured to be applied to the back of a subject's neck; wherein the first thermal contact is coupled to a first heating/cooling sources and the neck thermal contact is coupled to a second heating/cooling source; and a controller coupled with the first and second heating/cooling sources, wherein the controller is configured as a multi-channel controller configured to independently deliver thermal treatment to each of the first thermal contact and the neck thermal contact, wherein the thermal treatment comprises a temperature change of between 0.1 and 5 degrees C.

The first and second heating/cooling sources may each include a thermoelectric cooler (TEC). The controller may be configured to apply a drive signal comprising a pulsed signal having a frequency of greater than 200 Hz to the first heating/cooling source. The hydrogel may be as described herein, including a silicone hydrogel.

The apparatus may include one or more thermal sensors configured to determine a temperature within the ear canal.

The controller may be configured to deliver the thermal treatment for a predetermined time period, such as for between about 0.2 minutes and 30 minutes. Any of these apparatuses may include a speaker configured to emit audio signals into the first ear canal concurrent with the treatment (e.g., thermal, electrical, etc. signals). The controller may be configured to drive the speaker to emit a tone in conjunction with the thermal treatment.

The controller may be configured to cool the first thermal contact.

In general, a controller may include one or more processors, microprocessors, memories, microcontrollers, etc. for controlling operation of the apparatus, including generating the signals to be applied by the contacts and causing them to be applied.

As mentioned above, also described herein are methods of improving cognition, such as methods of improving attention and apparatuses for performing these methods. For example, a method of improving attention may include: identifying a need for enhanced attention to an event occurrence; and triggering a sensory input within a subject's ear canal and on a back of the subject's neck within 1-7 seconds of the occurrence of the event, wherein the sensory input comprises one or more of: an electrical stimulation, a thermal stimulation, a mechanical stimulation.

In general, the identification of the need for attention may include manually identifying the need for enhanced attention to the event occurrence. Alternatively, identifying the need may comprise automatically (or semi-automatically) identifying the need for enhanced attention to the event occurrence. For example, identifying the need may comprise detecting a lack of attention in the subject based one or more of: electroencephalogram (EEG) data, eye tracking data, and electrooculographic signal (EOG). The need may be manually identified (or semi-automatically identified) by a third party (or in some variations, the user) triggering a need for additional attention by predicting when the third party would like the subject to pay additional (or particular) attention. In some variations a computer system may be configured to, based on contextual input, such as identifying key phrases or words from a spoken conversation, that the subject should pay additional attention.

In some variations, the electrical stimulation, thermal stimulation, and/or mechanical stimulation within the subject's ear canal and/or cervical region (e.g., neck) is triggered within 2-5 seconds of the occurrence of the high-attention event (e.g., 2-5 seconds before the additional attention is desired). This may allow time for the subject to process and respond to the stimulation.

Triggering the electrical stimulation, thermal stimulation, and/or mechanical stimulation may include triggering a sub-sensory threshold stimulation. For example, triggering the stimulation within the subject's ear canal may comprises applying a temperature change of between about 0.1 and 5 degrees (e.g., between 0.1 and 3 degrees, between 0.1 and 2 degrees, between 0.1 and 1 degree, etc.). In any of these methods and apparatuses, the stimulation may include thermal stimulation, such as modulating the temperature at a frequency of greater than 300 Hz to generate a change in temperature of between 0.1 and 5 degrees C. (e.g., between 0.1 and 4 degrees C., between 0.1 and 3 degrees C., between 0.1 and 2 degrees C., between 0.1 and 1 degree C., etc.). The sensory input may comprise electrical stimulation, the electrical stimulation may comprises a biphasic, pulsed signal having a frequency of greater than 200 Hz (e.g., 250 Hz or more, 300 Hz or more, etc.) so that the wall of the first ear canal receives a current density of greater than 2 mA/cm².

In any of these variations the sensory input may be varied, e.g., by changing the sensory input every 10-60 seconds following triggering. In some variations, the method may further include switching the sensory input between one or more different locations within the subject's ear canal or outer ear every 10-60 seconds following triggering. Thus, any of these methods may include switching the sensory input between one or more of electrical stimulation, a thermal stimulation, and a mechanical stimulation every 10-60 seconds following triggering. In general, triggering may include applying the sensory input within both of a subject's ear canals or within just one of the subject's ear canals. Any of these methods may include switching the sensory input between one or more different locations within the subject's ear canal or the back of the subject's neck every 10-60 seconds following triggering. Any of these methods may include switching the sensory input between one or more of electrical stimulation, a thermal stimulation, and a mechanical stimulation every 10-60 seconds following triggering. In general, triggering may include applying the sensory input within both of a subject's ear canals and the back of the subject's neck.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A and 1B illustrate afferent and efferent nerve circuitry of the cervical plexus. In FIG. 1A, the schematic illustrates a diagram of the major peripheral nerves comprising the cervical plexus. Besides cervical spinal nerves C1-4 there are several cranial nerves (CN's) associated with the cervical plexus including CN IX (accessory nerve), CN X (vagus nerve), and CN XII (hypoglossal nerve). The cervical plexus also shares robust convergent inputs with CN V and the spinal nucleus of the trigeminal sensory complex. The efferent pathways regulate descending motor activity while the afferent pathways provide strong ascending sensory inputs to key arousal centers of the brain located in the pons and midbrain of the brain stem. The Apparatuses and methods described herein target the cervical plexus and associated circuitry as depicted. In order to target afferent pathways that do not transmit pain to the brain and to bypass efferent neuromuscular stimulation, the apparatuses and methods described herein may deliver pulsed waveforms or current waveforms having high-frequency. In FIG. 1B, the illustration shows the cervical plexus and associated peripheral nerve circuitry targeted by methods and devices described herein.

FIGS. 2A-2D illustrate dorsal anatomical perspectives of peripheral nerves targeted by craniocervical modulation as described herein. In FIGS. 2A-2C, the panel of images show the C2-C4 region of the neck; FIG. 2A shows regions targeted by four equally sized, 5 cm2 round electrodes illustrated as circles (not drawn to exact scale). The placement of devices as shown may modulate the activity of peripheral nerves of the cervical plexus, neck, and back of the head like the greater occipital nerve and lesser occipital nerve as shown. FIG. 2D illustrates the peripheral nerves comprising the cervical plexus and neck regions shown at a higher magnification. These nerves may be targeted by the apparatuses described herein and are each highlighted from left to right: hypoglossal nerve (201), great auricular nerve (203), lesser occipital nerve (205), spinal nerve C2 (207), and phrenic nerve (209); transverse cervical nerve 211, accessory nerve to the trapezius muscle 213, and spinal nerve C1 215, spinal nerve C3 217, and spinal nerve C4 219.

FIGS. 3A-3D show ventral anatomical perspectives of peripheral nerves targeted by the craniocervical modulation apparatuses (e.g., devices and systems) described herein. These images illustrate the peripheral nerves comprising the cervical plexus and neck regions shown from a ventral perspective. These groups of nerves or nerve bundles targeted by the apparatuses herein (e.g., the cervical applicator of the apparatus) are each indicated: cervical plexus 301 and vagus nerve 303; greater auricular nerve 305, hypoglossal nerve 307, transverse cervical nerve 309, and phrenic nerve 311.

FIGS. 4A-4D illustrates afferent neural pathways of the cervical plexus ascending through the trigeminal sensory nuclear complex to the brain stem and higher brain regions. In FIG. 4A, the illustration shows the trigeminal sensory nuclear complex (TSNC) shown as shaded regions in the spinal cord and brain stem. The TSNC is composed of the principal or primary nucleus receiving inputs from the trigeminal nerve (CN V) and its branches (V1-V3). The spinal nucleus of the TSNC receives inputs from C1-C3 spinal nerves, as well as other cranial nerves such as the facial nerve (CN VII), accessory nerve (CN IX), vagus nerve (CN X) and hypoglossal nerve (CN XII). From the TSNC, the afferent information carried by these peripheral nerves is transmitted ultimately through the ventral posteromedial (VPM) region of the thalamus and sensory cortex as shown in greater detail in FIG. 4B. The illustration in FIG. 4C shows where the cervical plexus and some trigeminal nerves enter the spinal nucleus of the TSNC in relation to the spinal cord, pons and brain stem. With greater anatomical detail the principal and spinal nuclei of the TSNC are shown in relation to other interconnected regions of the spinal cord and brain stem in FIG. 4D. The regions in FIG. 4D that represent sensory processing regions is indicated 405 while the regions 407 that represent motor nuclei are also shown. There is a significant degree of cross talk between the inputs of the TSNC and other key nuclei receiving similar primary cranial and cervical nerve information. These regions illustrated include the nucleus ambiguous and nucleus of the solitary tract. The TSNC provides direct outputs to the thalamus as shown in FIG. 4B, as well as to the locus coeruleus, raphe nuclei, and other major arousal centers of the brain.

FIGS. 5A-5C illustrate sensory inputs that gate arousal and regulate sleep-wake cycles by influencing the activity of the ascending reticular activating system and its neuromodulatory effectors. In FIG. 5A, two complimentary and opposing arousal neuromodulatory systems of the brain are illustrated. The ascending reticular activating system (RAS), which is responsible for gating arousal (wakefulness/alertness) and coordinating attention in response to incoming sensory stimuli is illustrated 501. Conversely the regions indicated by shading 503 are responsible for triggering sleep onset. One of the chief nuclei of the RAS that is illustrated is the locus coeruleus (LC), which receives inputs from many integrative sensory nuclei in the brain stem including the trigeminal sensory nuclear complex (TSNC). In FIG. 5B, the LC produces norepinephrine or noradrenaline (NA) in response to incoming sensory stimuli including from the TSNC. The plot illustrates how attention and general task performance relate to LC activity and NA levels. Low LC activity and NA concentrations correspond to low levels of arousal or relaxed, disengaged states. On the other hand, high LC activity and concentrations of NA may correspond to a high arousal or stressed states that produce high levels of distractibility and poor performance. In FIG. 5C, the schematic illustrates the major brain circuitry responsible for triggering and regulating sleep states. Components of the RAS including the LC, raphe nuclei (R), and pedunculopontine tegmental (PPT) nuclei that produce NA, serotonin (5-HT), and acetylcholine (ACh) respectively, and play crucial roles in maintaining wakefulness (arousal/alertness/attention) by suppressing the activity of other brain regions that in turn serve as sleep trigger centers by inhibiting RAS activity under appropriate conditions. This is known as mutual inhibition and may provide the basis of the “flip-flop” model of sleep-wake transitions. Affected as illustrated by the arrows in the schematic above, a critical event required for sleep onset is the suppression of LC activity and inhibition of NA signaling.

FIGS. 6A-6B illustrates sleep-wake cycles that are tightly regulated by RAS activity and opposing inhibitory network interactions. In FIG. 6A, the brain image shown on the left shows anatomical circuitry of the ascending reticular activating system (RAS) including the locus coeruleus (LC), pedunculopontine tegmental nuclei (PPT), and raphe nuclei (RN). These regions serve to establish and maintain conscious awareness, as well as attention to stimuli and arousal during wakefulness by transmitting noradrenaline (NA; norepinephrine), acetylcholine (ACh), and serotonin (5-HT) to vast regions of the brain from the LC, PPT, and RN respectively. The tuberomammillary nucleus (TMN) also utilizes histamine in a similar manner to regulate arousal during wakefulness. Another neurohormonal signal that stimulates these arousal regions is orexin (ORC). When the arousal brain centers like the LC are active during wakefulness, they inhibit the activity of sleep triggering neurons located in the ventrolateral preoptic area (VLPO) as shown by the diagram at right. In FIG. 6B, the image of the brain on the left shows anatomical circuitry and relationships whereby VLPO neurons inhibit LC activity, as well as the activity of other arousal circuits like the TMN and RN to trigger sleep onset. As illustrated by the diagram on the right, when neurons of the LC and other arousal regions decrease, the activity of VLPO neurons increase and begin to actively suppress the activity of arousal regions. This process is known as mutual inhibition and serves as the basis for the all or none nature flip-flop model of sleep-wake cycles.

FIGS. 7A-7C illustrate neuronal and neurovascular structure of the outer ear. In FIGS. 7A and 7B, the innervation of the pinna and external ear includes several nerves including branches of cranial nerve V (trigeminal nerve), cranial nerve VII (facial nerve), and cranial nerve X (vagus nerve), as well as the greater auricular nerve and auriculotemproal nerve, which are branches of the cervical plexus (spinal nerves C2 and C3) and illustrated. These nerves carry motor signals and sensory signals from and to the brain respectively. Several of these nerves provide communication between the external world and key arousal regions of the brain stem including the reticular nucleus, locus coeruleus and nucleus of the solitary tract. In FIG. 7C, the image shows that the outer ear is a highly vascularized structure to provide oxygen to the nerves through neurovascular coupling. The activity of nerves influences the activity of blood vessels and capillary and the activity of blood vessels and capillaries can also affect the activity of nerves. These dynamics can influence larger changes in vascular activity including blood pressure and heart rate given the tight coupling of the trigeminal and vagus nerves to cardiovascular and cerebrovascular activity. Through vascular signaling mechanisms and vascular coupling the modulation of local blood flow in the ear by pulsed electrical stimuli having a frequency higher than about 200 Hertz can exert an effect on cochlear blood flow through the actions of peripheral neuromodulation described by the present invention to affect hearing processes.

FIG. 8 illustrate neuromodulation of cranio-cervical networks utilizes known pathways from the external ear, face, head, and neck; any of these networks may be targeted and/or modulated as described herein. In some variations these networks may be modulated to influence auditory gain mechanisms and composite auditory processing. The methods and apparatuses described herein may introduce neurosensory bias by interfacing with cranial nerves (CN) and cervical nerves through an apparatus (e.g., device or system) that includes a multi-channel external ear stimulating electrode or MEESE. The external ear provides a unique opportunity to modulate several distinct sensory nerve pathways of cranial and cervical origins with high frequency pulsed stimuli having a primary frequency greater than about 250 Hertz and less than about two kilohertz or higher as described elsewhere in other embodiments of the invention. One particular aspect of the invention includes a bipolar MEESE design for modulating the trigeminal nerve (CN branch V3 as shown above) and the vagus nerve (CN X) from within the external acoustic meatus. The neuromodulation interface described herein may also include a design which enables an additional MEESE channel and contact sites for simultaneously modulating the activity of the greater auricular nerve (C2-C3) in targeting the pinna and the facial nerve by targeting the concha. Modulating these sites may alter composite auditory processing or the perception of sound by altering local blood flow of the ear including cochlear blood flow, as well as by altering or modulating the filtering properties and contributions of neural activity in certain brainstem nuclei like the locus coeruleus on auditory gain control.

FIG. 9A shows external portions of the ear (pinna) that may be optionally targeted instead or in addition to the ear canal. FIG. 9B shows an enlarged view of the ear canal (external acoustic meatus) region.

FIG. 10A illustrates one example of an apparatus (e.g., a neuromodulation device, system, or interface) that may be used for non-invasive auricular and cervical stimulation to modulate multiple integrated neural networks. FIG. 10B shows an example of one embodiment configured as a Multi-channel External Ear Stimulating Electrode (MEESE), e.g., a neurosensory biasing system, that may be used by the system of FIG. 10A to treat a subject. The MEESE or neurosensory biasing interface may include a multi-function transdermal neurostimulation system or neurosensory biasing system that may be combined with a storage base as illustrated; the base may also be configured to deliver punctate neurostimulation. Neurostimulation channels and active electrode regions are depicted by +/− or CH. The portion of the electrode interface designed to insert in the external acoustic meatus (top left) is shown as a bipolar electrode to stimulate the vagus and trigeminal nerves simultaneously. The MEESE shown on the top right includes a second neurostimulation channel; this second channel may be a bipolar electrode consisting of anode and cathode intended to stimulate the concha. Channel one and Channel two of the MEESE can operate in phase or out of phase of one another at the same frequency or at different frequencies to exert an effect on cochlear blood flow and other neurovascular dynamics of the ear. The stimulus may influence auditory perceptions before, after, or during neurosensory biasing, scrambled neuromodulation, or punctate neuromodulation using the device. The sites of MEESE electrodes shall be made of conductive polymers (including hydrogels) of material capable of comfortably delivering pulsed currents to the skin of the outer ear.

FIG. 10C shows an example of contacts that may be included as part of the system of 10A.

FIG. 11 illustrates the use of one example of a multi-function neurosensory biasing system as described herein. An MEESE can be stored in a base unit containing an additional neurostimulation channel in its base to achieve transient punctate or focal neuromodulation of nerve sites, as shown, on the head, face, neck, ear, or wrist to influence composite sensory processing. The storage base contains additional microprocessors and microcontrollers for communicating with the MEESE interface, but the base itself can also act as a neurostimulator. The system is similar to that shown in FIG. 10A, and is shown here with application to nerve targets on the head and face. The multi-functional aspects of the punctate neuromodulation may be used to treat or alleviate acute pain of the face, head or neck including pain due to headache, tempromandibular joint disorder, and motor disorders of the head, neck, and face. These disorders and the psychophysiological effects of them can indeed influence composite sensory processing so an embodiment of the multi-function neurobiasing system is to achieve an effect on sensory perception by being able to modulate a plurality of nerve locations as shown either in both a wearable form (MEESE) and a handheld form (storage base stimulation unit as shown).

FIGS. 12A-12C show another example of an apparatus (e.g., including an MEESE), configured as wearable headphones (earpieces) and neckpiece. FIG. 12A shows the two earpieces and neckpiece of the apparatus; FIG. 12B shows possible configurations of the electrical contacts. FIG. 12C shows one earpiece of the apparatus worn in an ear. In this example, the apparatus includes two bipolar stimulating electrodes per headphone where one channel is designed to stimulate the external acoustic meatus and the other channel designed to stimulate the ear lobe in order to modulate the activity of the vagus and/or trigeminal nerves and the greater auricular nerve (e.g., C2-C3) simultaneously or nearly simultaneously. The neckpiece may stimulate the cervical nerves. The portion of the headset designed to deliver pulsed electrical stimuli may be made of a conductive polymer like a conductive silicon or conductive urethane or other biocompatible conductive material; in particular, the material may be a conductive hydrogel. MEESE electrodes may be constructed using non-conductive insulating layers (NIL) as shown and can be made of non-conductive materials acting as a shim between active bipolar electrode regions. The components may be connected wirelessly (as shown) or via a wired connection. Components are not shown to scale.

FIGS. 13A-13B illustrate another example of an apparatus as described. FIG. 13A shows the application of the apparatus to a subject. FIG. 13B shows an enlarged view of an earpiece of the apparatus.

FIG. 13C is a schematic illustration of one example of an apparatus (such as the one shown in FIG. 13A) that may be used with the methods described herein.

FIGS. 14A-14D illustrate examples of different variations of apparatuses as described herein. In FIG. 14A the apparatus is configured as a device for punctate electrical neurosensory biasing and includes an external battery. This variation may be referred to as an EPEN (electrical peripheral epidermal neurosensory) device. FIG. 14B is a similar apparatus also configured to deliver punctate electrical neurosensory signals (e.g., neurosensory biasing signals) and includes an internal battery. The device shown in FIG. 14C is similar, and includes an ultrasound transducer on the base portion that may be used to deliver mechanical and/or thermal signals (configured as an ultrasonic peripheral epidermal neurosensory device). FIG. 14D shows a variation of a device for punctate electrical neurosensory biasing having a sleeve/tube on the body.

FIG. 15A shows one example of an electrical contact that may be part of a neck contact (e.g., neckpiece) or an EPEN device (e.g., button electrode) as described herein, having a highly uniform current distribution. FIGS. 15B and 15C illustrate a current distributing layer (e.g., Ag/AgCl layer) that may be used as part of the electrical contact shown in FIG. 15A.

FIGS. 16A-16D illustrate examples of an electrical contact such as the one shown in FIG. 15A having regions of current (e.g., channels).

FIGS. 17A-17B illustrate another example of a pattern, shown as an electro-micro grid, for an electrical contact resulting in more uniform current distribution.

FIGS. 18A-18B illustrate another example of a pattern, shown as an electro-nano dots, for an electrical contact resulting in more uniform current distribution.

FIGS. 19A-19B illustrate another example of a pattern, shown as a star-shaped pattern, for an electrical contact resulting in more uniform current distribution.

FIG. 20A illustrates the use of an apparatus such as the EPEN (FIG. 14A) or UPEN (FIG. 14C) to perform one or more cosmetic treatments. FIG. 20B illustrates target regions for punctate treatment similar to that shown in FIG. 20A.

FIG. 21 illustrates the use of an apparatus such as the EPEN (FIG. 14A) or UPEN (FIG. 14C) to provide neurosensory biasing for treatment of blepharospasm.

FIG. 22 illustrates the use of an apparatus as described herein configured to provide neurosensory biasing of median nerve fibers.

FIG. 23 illustrates the use of an apparatus as described herein for treatment of motion sickness.

### DETAILED DESCRIPTION

The methods and apparatuses described herein generally relate to non-invasive neuromodulation. In particular, these apparatuses (devices, systems, etc.) may relate to non-invasive auricular and cervical neurostimulation to modulate one or more networks of nerves. For example, these methods and apparatuses may modulate nerves located at the base of the skull or in the neck, such as craniocervical nerves, cranial nerves, or cervical spinal nerves and/or the auricular nerve for influencing one or more biological homeostasis and related physiological processes. Also described are methods and apparatuses for improving cognitive function, including directing attention. Finally, also described herein are methods and apparatuses that may be used for cosmetic treatments, treatment of anxiety, motion sickness, carpal tunnel, or neuropathic pain.

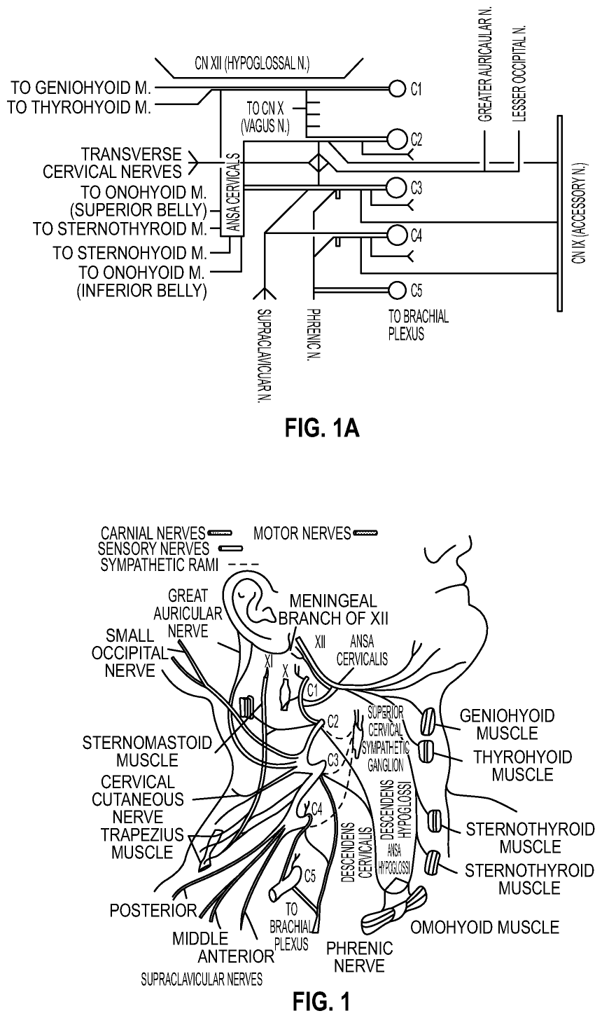

The cervical plexus is a collection of nerves located at the base of the skull running down the neck from cervical (C) vertebrae 1-4 (C1-C4; FIG. 1). The cervical plexus may be targeted using systems and devices mounted to the surface of the skin of an individual, worn by an individual, or making contact with the individual when resting in a chair, on a cot or in a bed for example. The apparatuses and methods described herein may be used to modulate the activity of peripheral nerves to affect brain regions or neural circuits involved in metabolic and homeostatic regulation of appetite, sleep, body weight or body temperature. Without being bound by theory, the methods and apparatuses described herein may modulate the neural circuits and their major actions by modulation of craniocervical activity.

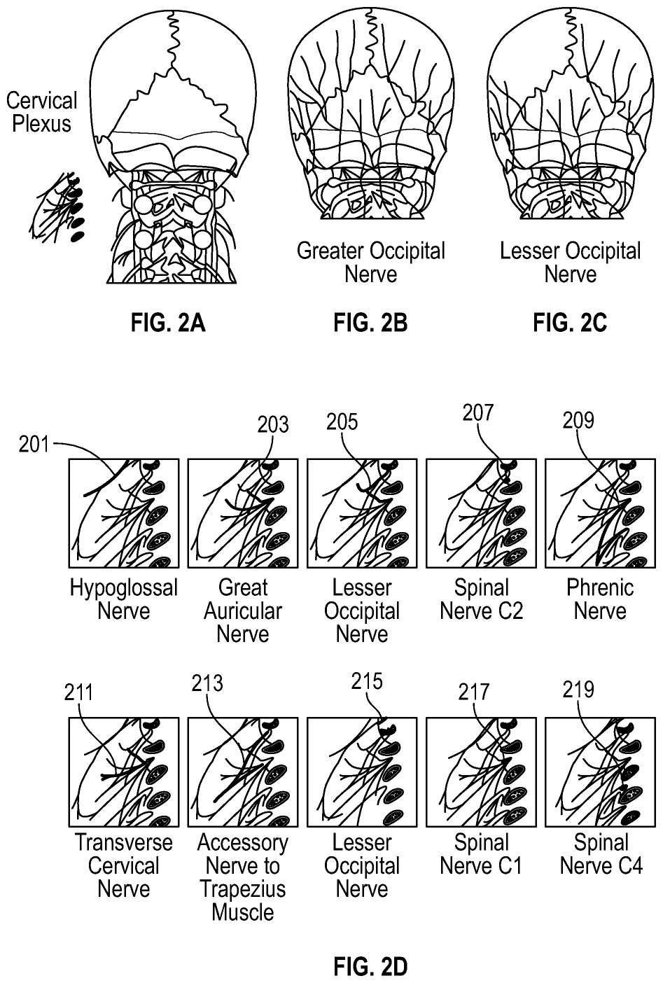

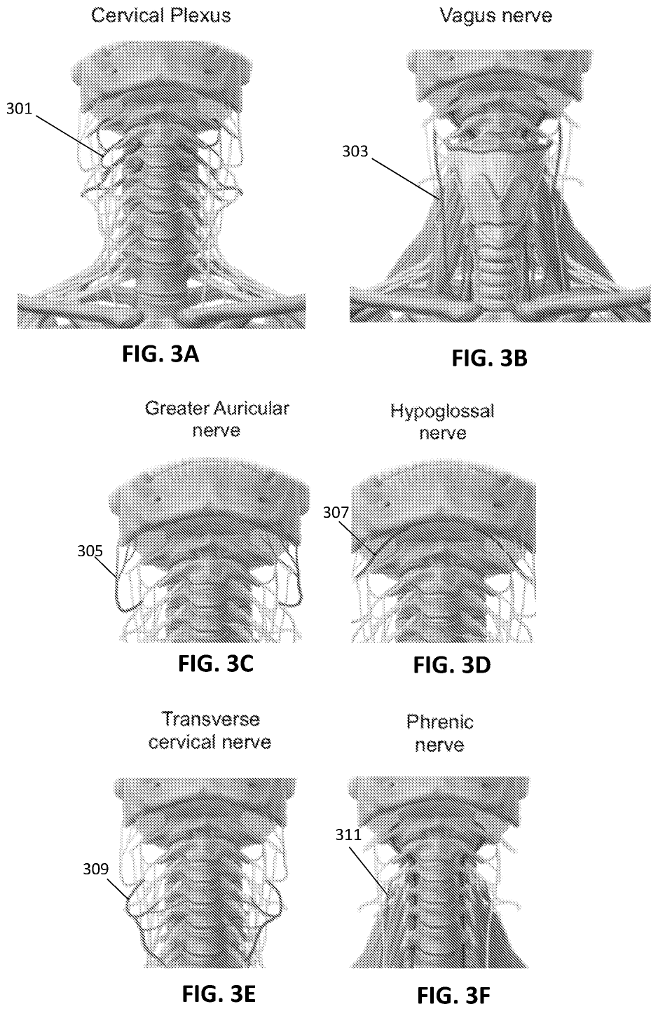

Each of the cervical nerves forming the cervical or craniocervical plexus communicates with one another in a superior-inferior fashion close to their origins, thus C1 receives communicating fibers from C2, C3 from C2, and C4 from C3. These communicating fibers are contributions from the sympathetic trunk (sympathetic nervous system) to the cervical plexus. These fibers are gray rami (meaning blood vessel accompanied) descending from the superior cervical ganglion (the largest of the three cervical ganglia; FIG. 1B). With the exception of C1, each cervical branch divides into an ascending branch and a descending branch, and unite with branches of the adjacent cervical nerve to form loops, for example, the loop formed between C2 and C3 that contributes branches to the ansa cervicalis (FIG. 1B). Those loops and the branches from them comprise the cervical plexus that may be targeted by the apparatuses and methods described herein as shown in greater detail in FIGS. 2A-2D and FIG. 3.

As seen in FIG. 1B, branches of the cervical plexus include mixed motor fibers innervating muscles and sensory cutaneous nerves innervating the skin of the anterolateral neck, the superior part of the thorax (superolateral thoracic wall) and scalp between the auricle (pinna; outer ear) and the external occipital protuberance located at the base of the skull.

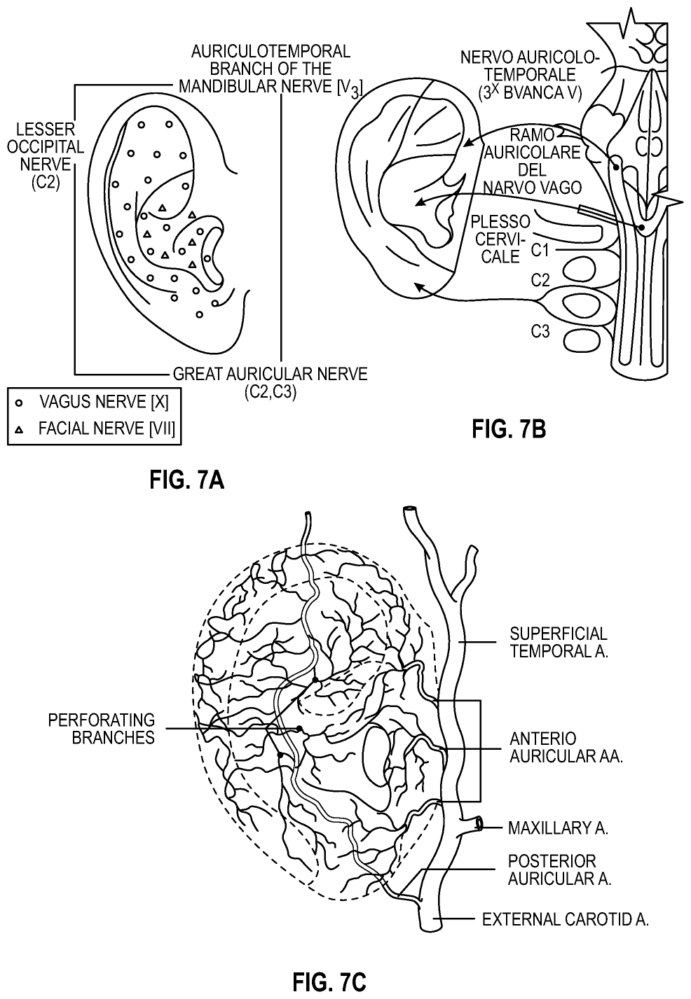

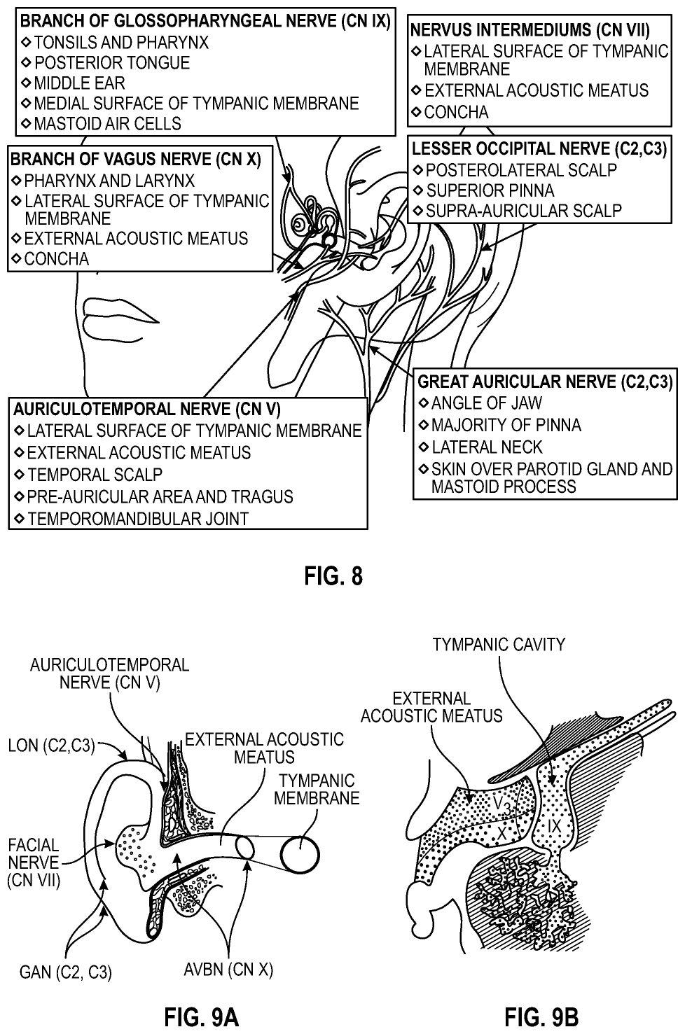

The auricle or external ear is a complexly shaped structure, innervated by a dense plexus of sensory afferents that transmit somatosensory, proprioceptive, and neurovascular information to the brain as illustrated in FIGS. 7 and 8. Auditory sensations and perceptions results from integrated sensory inputs (for example, somatosensory and proprioceptive inputs) that include classical hearing mechanisms where sound pressure affects hair cells, the cochlea, and tympanic membrane. In addition to environmental stimuli, hearing and the processing of auditory information is significantly influenced by internal physiological systems or states, such as blood pressure or the activity of neurovascular circuits that include neurons, glial cells, and capillaries or blood vessels (e.g., the neurovascular triad).

Traditional TENS devices operate at a stimulus frequency of about 80 to 130 or 50000 cycles per second (Hertz or Hz), which is an optimal frequency band for stimulating neuromuscular activity. Such TENS devices may operate at frequencies from 350 to 50000 Hz, which avoids (or mildly suppresses) neuromuscular activation while enabling modulation of afferent sensory and proprioceptive fibers. Likewise, the use of 350 to 50000 Hz pulsed transdermal electrical stimulation (pTES) waveforms minimizes or eliminates the activation of pain fibers and pathways, which typically respond up to about 200 Hz. Therefore, the choice of pTES frequencies provides a safer and more comfortable experience for users by minimizing muscle stimulation and pain fiber activation compared to traditional TENS and other substantially equivalent approaches like electrical muscle stimulation (EMS), neuromuscular electrical stimulation (NMES), and powered muscle stimulation (PMS).

The afferent sensory circuits making up the cervical plexus rises first through the spinal cord, medulla, pons, and midbrain to higher brain regions; the methods and apparatuses described herein may stimulate or trigger endogenous neurophysiological processes to alter the activity of the autonomic nervous system by targeting some or all of these circuits.

The sensory (posterior or cutaneous) branches of the cervical plexus emerge around the middle of the posterior border of the sternocleidomastoid muscle (roughly the midpoint on the side of the neck located towards the back of the head in line with the back of the ear). This area is clinically significant and recognized as the nerve point of the neck, where anesthetics can be injected to achieve cervical nerve blocks to alleviate head pain (including headache), neck pain, face pain, tooth pain, and shoulder pain, for example. In some variations the apparatuses described herein may be positioned on the back of the neck in between the locations of these nerve points as illustrated in FIG. 2A.

There are four main pairs of sensory branches of the cervical plexus originating from the two loops formed between the ventral rami of C2 and C3, and C1 and C4 (as shown in FIGS. 2A-2D and 3). The branches of the loop between C2 and C3 are: the Lesser Occipital nerve (formed by C2); the Great Auricular nerve (formed by C2 and C3); and the Transverse Cervical nerve (formed by C2 and C3). The branches of the loop between C3 and C4 are the Supraclavicular nerves (formed by C3 and C4).

The Lesser occipital nerve is formed by the second cervical nerve (C2) only, and courses to supply the skin of the neck and the scalp posterosuperior to the clavicle (FIG. 3A). The Great Auricular Nerve is the sensory branch, which originates from the C2 and C3 nerves. It courses upwards in a diagonal fashion and crosses the sternocleidomastoid muscle onto the parotid gland, where it divides and innervates the skin over the parotid gland, the posterior aspect of the auricle, and an area of skin extending from the angle of the mandible of the mastoid process (FIGS. 2A and 3).

The Transverse cervical nerve is formed by axons from the C2 and C3 nerves. It supplies the skin covering the anterior triangle of the neck (FIGS. 2B and 3). The Supraclavicular nerve is formed by C3 and C4 nerves and emerges as a common trunk under cover of the sternocleidomastoid muscle. It sends small branches to the skin of the neck. Some of those branches (supraclavicular) also cross the clavicle to supply the skin over the shoulder (FIGS. 1B and 3). Besides these main sensory branches of the cervical plexus, as illustrated in FIGS. 1 and 2-5 there are several sensory components of cranial nerves interconnected within the plexus including CN V (trigeminal nerve), CN IX (accessory nerve), CN X (vagus nerve), and the CN XII (hypoglossal nerve). The phrenic nerve also transmits sensory information from the diaphragm through the cervical plexus to the brain as discussed below.

The motor branches of the cervical plexus form the ansa cervicalis, which is a nerve loop innervating the infrahyoid muscles in the anterior cervical triangle, and also form the phrenic nerve which supply the diaphragm and the pericardium of the heart (FIGS. 1A-1B, 2B, and 3). The ansa cervicalis loop is made of five pairs of motor nerve branches originating from C1 to C3 nerves to supply the infrahyoid muscles in the anterior cervical triangle. The motor nerves forming the ansa cervicalis are Geniohyoid nerves (C1), Thyrohyoid nerves (C1), Omohyoid nerves (C1-C3), Sternohyoid nerves (C1-C3), and Sternothyroid nerves (C1-C3; FIG. 1).

The phrenic nerve originates chiefly from C4, but also receives contributions from C3 and C5 (FIGS. 1B, 2B, and 3). It is formed at the superior part of the lateral border of the anterior scalene muscle, at the level of the superior border of the thyroid cartilage.

The phrenic nerve contains motor, sensory, and sympathetic nerve fibers. It provides the sole motor supply to the diaphragm and receives sensory information from its central region. In the thorax, the phrenic nerve innervates the mediastinal pleura and pericardium of the heart. The phrenic nerve descends obliquely across the anterior scalenus muscle, deep to the prevertebral layer of deep cervical fascia and the transverse cervical and suprascapular arteries. It runs posterior to the subclavian vein and anterior to the internal thoracic artery as it enters the thorax.

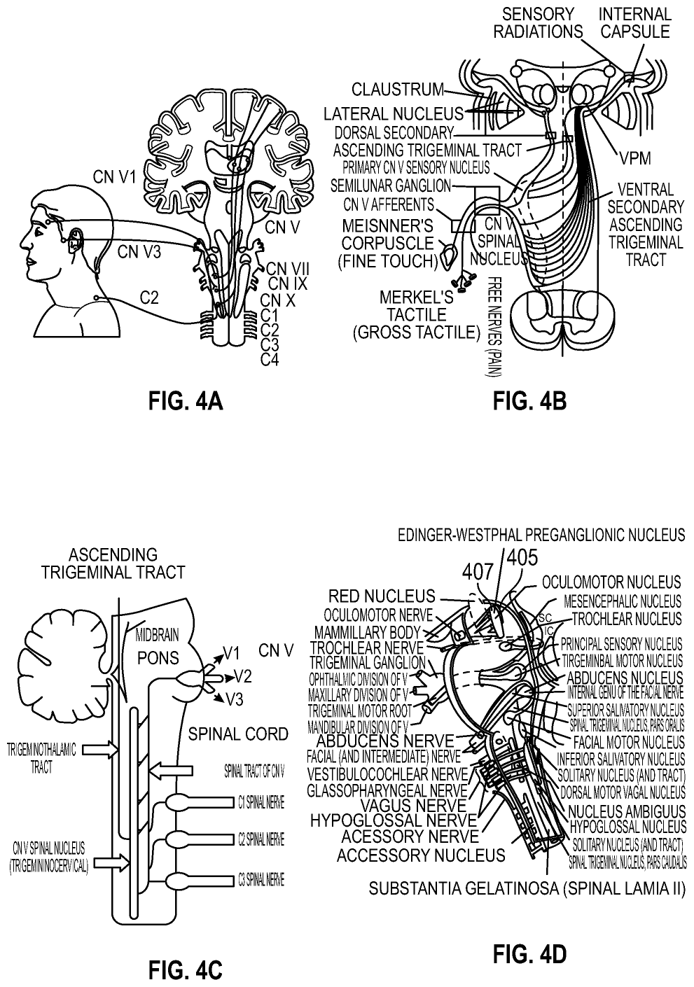

As illustrated in FIG. 4 the trigeminal sensory nuclear complex (TSNC) receives monosynaptic inputs from the cervical plexus and trigeminal nerves. In turn the TSNC transmits information to collateral and ascending pathways to multiple brain regions that regulate arousal and coordinate neurobehavioral engagement with the environment, such as the thalamus, the superior colliculus, the cerebellum, and several regions of the ascending reticular activating system (RAS) including the locus coeruleus (LC) and pedunculopontine tegmental nuclei.

The RAS is a collection of nuclei and circuits that sort, filter, integrate, and transmit incoming sensory information from the brain stem to the cortex to regulate sleep/wake cycles, arousal/alertness, attention, and sensorimotor behaviors. The endogenous neuromodulatory actions of the RAS on consciousness and attention are orchestrated by at least three distinct sets of brainstem nuclei that include cholinergic neurons of the PPT, noradrenergic (NA) neurons of the LC, and serotonergic (5-HT) neurons of the raphe nuclei.

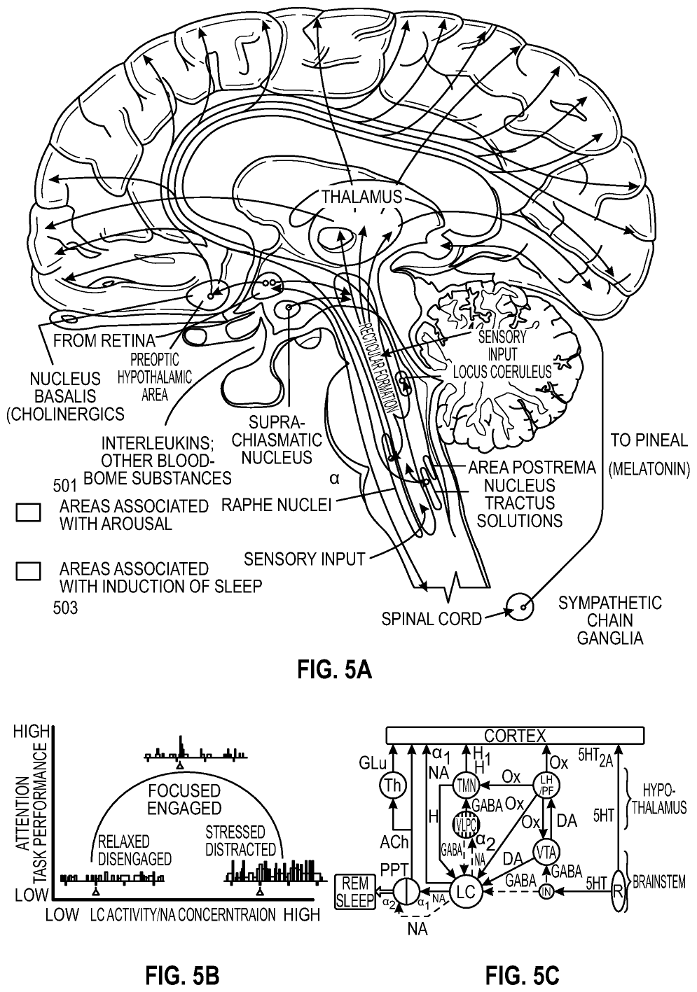

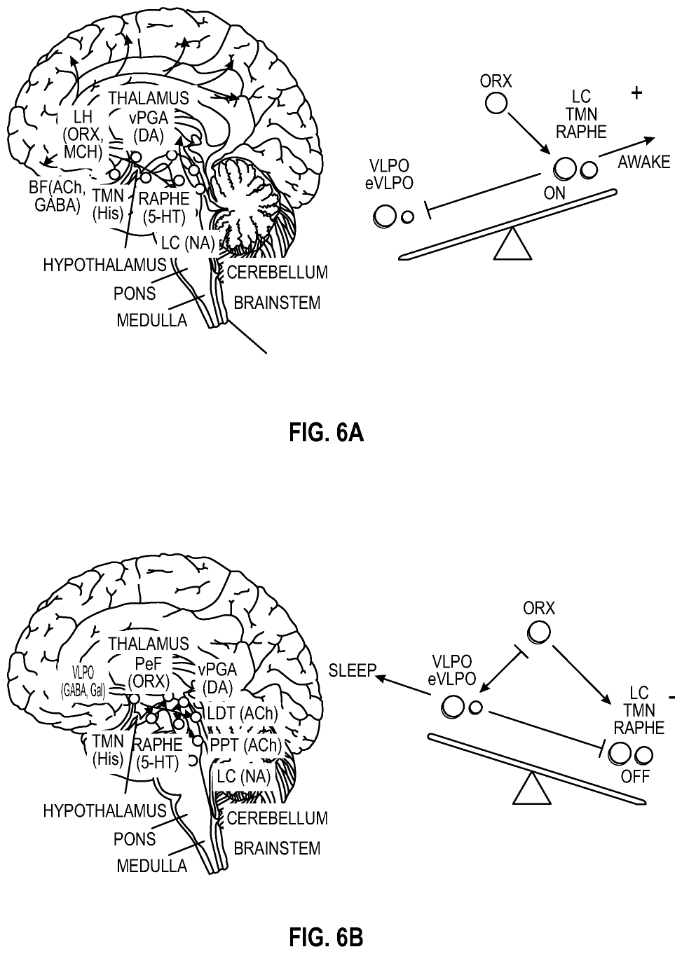

Through a network of connected brain stem nuclei in the pons and midbrain (FIGS. 5D and 6B), sensory inputs first act upon the brain to engage ascending RAS networks, which generate global arousal (“waking”), alerting, and orienting cues as parsed sensory information projects through thalamic pathways onto the cortex for additional processing and integration. More specifically RAS networks (including neurons of the LC, PPT, RN) act to gate information flow from the sensory environment to the cortex.

In an activity-dependent manner the RAS rapidly triggers neurobehavioral transitions across different states of awareness and consciousness. Depending on their firing rates for example, neurons of the PPT can differentially mediate REM sleep states and neurons of the LC can trigger sleep/wake transitions.

Disrupted activity of ascending RAS networks underlies several neuropsychiatric conditions and disorders, such as insomnia, anxiety, depression, post-traumatic stress disorder (PTSD), and attention deficit hyperactivity disorder (ADHD; Sara, 2009, Lemaire et al., 2014, Garcia-Rill et al., 2015, Gummadavelli et al., 2015). In fact there are numerous lines of evidence demonstrating insomnia is a “waking” disorder (hyperarousal) of RAS networks rather than a sleep disorder per se. Similar hyper-arousal hypothesis have also recently received support whereby PTSD, anxiety, some attention disorders are different manifestations of hyperadrenergic activity and/or pathologically high levels of sympathetic activity (for example, chronic stress).

The “flip-flop” model of sleep onset describes how RAS nuclei (LC, RN, and PPT) engage in mutual inhibition with other key brain nuclei to rapidly regulate conscious awareness across sleep-wake transitions (FIG. 6). As previously described, one of the primary functions of the LC is to keep the brain and body constantly prepared for or engaged in action by allocating and coordinating attention and arousal in response to all incoming sensory information first processed in the brain stem. It does so by transmitting the monoamine NA (norepinephrine; NE) to about 85% of the brain where it acts on different types of receptors including alpha (a) and beta (β) adrenergic receptor subtypes. LC activity and NE transmission act as real-time central regulators of baseline brain activity. They exert many dynamic filtering operations on brain activity that is required to optimize signal-to-noise relationships amongst neural networks for the conscious processing of incoming sensory information and awareness.

The essential alerting and orienting functions of the LC also influence sympathetic nervous system (SNS) activity and underlie the neurophysiological foundations of the commonly known “fight-or-flight” response. Under normal sensory processing these actions allow the brain and body to be engaged to perform general tasks as illustrated in FIG. 5B. However, if a threatening sensory stimulus is presented (for example, the sound of an alarm or sight of a vicious animal) then the activity of the LC surges to instantly prepare the body to escape or defend itself. Returning the body to calm or relaxed states after a fight-or-flight response, as well as from persistent ongoing processing of sensory stimuli (daily tasks; stress) typically takes considerably longer time than it takes to become aroused, attentive, or alert. This is why people generally require several tens of minutes to calm down after working or studying to relax enough and fall asleep. The deepest stages of sleep typically occur twenty or more minutes after sleep onset. Somewhat paradoxically and due to the intrinsic nature of flip-flop style mechanisms of sleep onset however, the transition from awake to sleep does occur within seconds. The major point is that suppression of LC and SNS activity is an essential requirement and critical trigger for sleep onset to occur (FIG. 6B).

Stimulation, suppression, and perturbation of LC activity via cervical plexus modulation of TSNC networks could all result in a shifting of the dynamically bi-stable brain networks responsible for triggering sleep onset that are further described below.

The LC suppresses sleep circuit activity during awake behaviors in response to sensory inputs (FIG. 6A). Conversely, LC activity is suppressed by the brain's sleep circuitry to trigger sleep onset (FIG. 6B). Other arousal networks are also involved in regulating sleep-wake behaviors. For example, the pedunculopontine tegmental (PPT) nucleus uses the neuromodulator ACh to play a crucial role in gating information flow between the thalamus and the cerebral cortex. PPT neurons are highly active during wakefulness and during REM sleep and experience low activation during NREM sleep.

The PPT neurons are regulated by mutual inhibition with sleep circuits as described below and illustrated in FIG. 6. The dorsal and median raphe nuclei (RN), ventral periaqueductal grey matter, and tuberomammillary nucleus (TMN) also each produces different neuromodulators that regulate arousal by acting on cortical and subcortical networks (FIG. 6). Neurons in the RN, ventral periaqueductal grey matter (vPAG), and TMN produce 5-HT, dopamine (DA) and histamine respectively. Interestingly, many anti-histamine drugs (Benadryl, for example) block the histaminergic arousing signal and cause sleepiness. Another collection of neurons in the lateral hypothalamus produce a neurotransmitter called orexin (ORX; also known as hypocretin), which directly stimulates LC, PPT, and RN arousal centers, as well as the cerebral cortex. It is believed that ORX serves as a key signaling molecule that integrates metabolic, circadian and sleep debt influences to determine whether an individual should be asleep or awake and active. The majority of so-called “sleep neurons” however are located in the ventrolateral preoptic area (VLPO). These sleep neurons are silent until an individual shows a transition from waking to sleep when the VLPO neurons become active (due in part to decreased LC activity) and suppress LC, TMN, and RN activity (FIG. 6B). The sleep neurons in the preoptic area receive inhibitory inputs from some of the same regions they inhibit, including the LC, TMN, and RN (FIG. 6). Thus, they are inhibited by NE, histamine, and 5-HT. This mutual inhibition is thought to provide a basis for establishing and triggering periods of sleep and waking.

Transitions between the bi-stable states of wakefulness and sleep occur relatively quickly, often in just seconds as mentioned. The neurological mechanisms that control these rapid transitions are thought to be analogous to a “flip-flop” electrical circuit. A flipflop in an electrical circuit can assume one of two states, usually referred to as “on” or “off”. Similarly, sleep neurons are either active and inhibit the wakefulness neurons, or the wakefulness neurons are active and inhibit the sleep neurons. Because these regions are mutually inhibitory, it is impossible for neurons in both sets of regions to be active at the same time. This flip-flop, switching from one state to another quickly, can be unstable and sensitive to perturbation. The same flip-flop analogy is also used to sometimes describe brain mechanisms involved in switching between REM sleep and NREM stages of sleep. Different neurotransmitters and different groups of neurons, such as ACh and NE from the PPT and LC respectively are involved in the transitions between REM and NREM sleep as previously mentioned (FIG. 5C).

The biochemical and neuroanatomical processes affected by craniocervical modulation are also known to affect inflammation and learning or cognition. Methods and systems described are also designed to modulate inflammation and cognition.

For example, described herein are non-invasive neural interfaces capable of dynamically and electrically suppressing or perturbing the ascending reticular activating system (RAS network) activity by modulating the activity of the cervical plexus and/or auricular nerves and providing an efficient, chemical-free approach to restoring poor daily function attributable to sleep loss, stress, or attention and mood disorders.

Devices described herein are configured to suppress and scramble the endogenous activity of RAS circuits including the locus coeruleus (LC) by modulating cervical plexus and cranial nerves in the dorsal region of the neck and/or by modulator auricular nerves of the ear and pinna. Cervical and cranial nerves, as well branches of nerves present in the ear and pinna, transmit primary sensory information directly to the trigeminal sensory nuclear complex (TSNC) and influence the activity of the LC and ascending RAS as discussed. By suppressing activity in these ascending arousal networks, the transdermal and peripheral neuromodulation waveforms can stimulate physiological relaxation, decrease appetite, boost metabolic activity, regulate the activity of metabolic pathways, regulate body temperature, and alter other biologically homeostatic processes.

These effects can be observed acutely as autonomic changes in heart rate (HR), shifts in heart rate variability (HRV), and increases in skin temperature (particularly facial, hand, and foot temperature). The effects may also accompanied by a slowing in respiration (perhaps due to effects on the phrenic nerve) and a general sense of psychological relaxation. Any of the methods and apparatuses described herein may include feedback (or confirmation) by detecting changes in all or some of these. Modulation of autonomic function using the craniocervical plexus and/or auricular nerves as target sites can alter the activity of hypothalamic nuclei involved in appetite, temperature, and circadian regulation.

As described herein modulation of the craniocervical plexus and/or auricular nerves involves the delivery of high frequency currents (typically higher than 200 cycles per second, such as higher than 300, 400, 500, 550, 700, 1000, 10000, or 50000 cycles per second) in a manner to increase comfort and maximize efficacy. Neuromodulation waveforms may include other features whereby the amplitude of currents are modulated in a temporal pattern such as amplitude modulation or where the frequency of stimulus patterns is further modulated through burst or frequency modulation. Currents transmitted to the skin typically will not exceed a peak of 35 milliamps and may maintain a current density less than about 5 milliamps per centimeter squared.

In some variations the devices described herein contain one or more pairs of electrodes for passing electrical current through the skin of the neck, shoulders, or skin overlying the base of the skull to alter autonomic function in a manner that influences biological homeostasis. For example, described herein are systems for regulating appetite by increasing the rate of satiety onset following the modulation of craniocervical nerves using pulsed electrical currents delivered transdermally to the skin of the neck, shoulders, or head. In some variations, the apparatuses may be connected using wireless communication protocols for adjusting the timing of stimulation to begin about 5 minutes before a meal, such as 7, 8, 9, 10, 15 or 20 minutes before a meal.

In some variations, the apparatuses may be used for modulating the activity of brain circuits such as hypothalamic circuits involved in temperature regulation. Such apparatuses may modulate the activity of craniocervical nerves before sleep or during sleep to alter body temperature.

Some variations of the systems described herein may incorporate electrodes into a chair or specifically into the neck region of a chair. Other embodiments may include the incorporation of electrodes and system controllers such as integrated circuits and current amplifiers, resistors, capacitors, wireless communication chips, and power supplies into a pillow, such as a travel pillow. These embodiments are designed to modulate the activity of craniocervical nerves, cranial nerves, or cervical nerves while a person is resting in a chair or on a sofa or bed.

In some variations, the apparatuses described herein may use pulsed ultrasound having an acoustic frequency less than about 100 megahertz and higher than about 20 kilohertz delivered through the skin of the neck to the nerves of the cervical plexus of the craniocervical plexus at an intensity greater than about 50 milli-watts per centimeter squared, such as 50 watts per centimeter squared, to affect the activity of nerves targeted in a manner that results in changes to homeostatic processes, such as sleep, appetite, metabolic activity, or body temperature.

The apparatuses and methods described herein may use blue light having a wavelength of about 480 nanometers or red light having wavelength of about 700 nanometers or a mixed combination of electromagnetic radiation having an optical wavelength from about 400 to about 1000 nanometers to alter the activity of the cervical plexus. Such embodiments may transmit light or photons from a device through the skin of the head or back of neck or the sides of the neck either alone or before or after or simultaneously with pulsed electrical stimuli or pulsed ultrasonic stimuli. Such optical based methods may alter craniocervical activity in a manner to influence autonomic functions by delivering greater than a few milli-watts of energy.

Any of the methods and apparatuses described herein may use artificial intelligence or computational methods to provide feedback to a user by way of known communication methods such as email, text, phone call or other method. The feedback may be constructed as such to reinforce desired behaviors such as adhering to a sleep schedule or eating specific foods or quantities of food. The system may deliver such digital content in a manner that is tied to geography, behaviors such as feeding or sleeping, times of day such as morning, afternoon, or night. Other cues in the environment or predicted by data collected from the system using integrated or connected sensors may be used to trigger messages or engage a messaging or alert system for the user.

The methods and apparatuses described herein may enhance skill training by delivering craniocervical neuromodulation protocols to the skin of the head, neck or shoulders in a manner that is timed in close temporal space to skills learned. Such embodiments may be used to enhance foreign language learning or to enhance skill development of sensorimotor skills. Other embodiments may be used to enhance or augment learning of information by modulating memory consolidation processes during sleep or after training.

The methods and apparatuses described herein include or be configured to communicate with other software applications to control the timing of information such that attention can be gated and modulated in a manner to optimize performance and problem solving. Such integration with human computer interfaces such as learning tools can be useful for accelerating the processing of human cognition or the rate at which information can be learned and retained.

The craniocervical neuromodulation systems and methods described herein may be useful for suppressing or enhancing for immunosuppression to treat inflammatory diseases, including autoimmune disorders and conditions, or to therapeutically enhance immune processes.

Stimulation of vagal and sympathetic pathways can affect inflammatory processes mediated by cholinergic and adrenergic receptors in immune tissues, including the spleen, thymus, and lymph nodes. Vagal stimulation has been shown to suppress inflammatory cytokines via nicotinic acetylcholine receptor subunit α7. Sympathetic stimulation via a T cell population that expresses beta-adrenergic receptors and which release of acetylcholine (ACh) can suppress immune activity by modulating macrophages expressing nicotinic receptors.

Thus, increased sympathetic nervous system activation suppresses immune activity and would be beneficial for treating disorders of overactive immune activity including autoimmune disorders. Similarly, increasing vagal nerve activation can suppress inflammatory processes including cytokine production. In other conditions, e.g. as a co-therapy for immune oncology to enhance immune targeting of cancerous tissue, increasing inflammatory processes and macrophage activation would be beneficial and/or therapeutic.

Systems and methods to bidirectionally modulate peripheral nerve pathways that control immune state, including inflammation (e.g. cytokines) and macrophage activation, would be beneficial to a broad range of disorders and diseases for which either increasing or decreasing the activation of the immune system would be serve as a prophylactic or therapy. However, existing peripheral stimulation systems, devices, and methods are lacking for modulating vagal and/or sympathetic nerve pathways using invasive and/or noninvasive neuromodulation techniques to control immune system activation and inflammation. Described herein are neuromodulation systems targeting craniocervical (or other peripheral) nerves to modulate immune function using one or more form of neuromodulation from the list including but not limited to: electrical stimulation (including electrical stimulation comprising one or more of: pulsed electrical stimulation, charge-balanced biphasic (i.e. AC) waveforms, transdermal techniques, and invasive (i.e. nerve cuff) techniques), ultrasound neuromodulation (including pulsed ultrasound neuromodulation, continuous wave ultrasound neuromodulation, ultrasound neuromodulation that causes neuromodulation by non-thermal mechanisms, ultrasound neuromodulation that causes neuromodulation by thermal mechanisms, and focused ultrasound neuromodulation (e.g. delivered by a phased array)), magnetic stimulation (i.e. TMS), optogenetic neuromodulation, RF neuromodulation, thermal neuromodulation, and other forms of neuromodulation.

Autoimmune disorders are numerous and the prevalence of these disorders in the general population have increased in recent years. The list of autoimmune disorders amenable for treatment by systems and methods for enhancing sympathetic and/or vagal nerve pathways to suppress inflammatory processes by neuromodulation of craniocervical (and/or other peripheral nerve targets) includes but is not limited to: Addison's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclerosing disease, Immune thrombocytopenic purpura (ITP), Inclusion body myositis (IBM), inflammatory bowel disease, Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis, Myasthenia gravis, Myositis, Narcolepsy, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III; Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Wegener's granulomatosis (or Granulomatosis with Polyangiitis (GPA)), shingles (for which autoimmune disorders are a risk factor), chronic spinal cord injury (not acute effects), sympathetic nervous system-related immunosuppression caused by hypertension, and acute idiopathic demyelinating polyneuropathy (AIDP; i.e. Guillain-Barré syndrome) in response to campylobacter infection.

In general, neuromodulation systems for modulating immune function by targeting craniocervical nerves (and/or auricular or other peripheral nerve targets) may include closed loop functionality for starting, stopping, or altering neuromodulation based on physiological, behavioral, or other measurements of a subject's biological state, which may include wearable and/or implantable sensors, non-contact physiological sensors, data from a blood test, location-based data, etc.

Vagal stimulation, including auricular vagal stimulation, suppresses inflammation and has been described as a potential therapy for sepsis and other inflammatory disorders. However, there remains controversy about the pathway from vagal nerves to sympathetic pathways that are required for immune suppression via the spleen. One hypothesis is that stimulating the vagal nerve addresses an imbalance of vagal/sympathetic tone, yet existing systems and methods for immune modulation by neural pathways has focused on vagal stimulation and neglected sympathetic pathways.

In one embodiment of the invention, a craniocervical and/or auricular neuromodulation system can affect both vagal and sympathetic pathways in a direction that supports the function of each of these pathways on the immune system. For example, electrical stimulation (or another form of neuromodulation) to a craniocervical and/or auricular target activates a vagal pathways (presumably directly via efferents due to observed reduction in a subject's heart rate) and inhibits sympathetic pathways (likely via brainstem afferents).

In some variations, the methods and apparatuses described herein may be useful for low frequency craniocervical and/or auricular neuromodulation during sleep. Systems and methods for low frequency craniocervical and/or auricular neuromodulation during sleep can improve the restorative quality of sleep and enable subjects to achieve improved mood and anxiolysis at wakening. Slow breathing is known to activate anxiolytic pathways but systems and methods for causing slow breathing before or during sleep do not exist. Neuromodulation of craniocervical pathways before or during sleep (including in a sleep-phase-dependent manner) at low frequency (i.e. 0.05 to 0.5 Hz; e.g. at about 0.2 HZ) can induce slow breathing and improve low-anxiety sleep and restorative physiology during sleep. Systems and methods for neuromodulation targeting peripheral nerve pathways (i.e., craniocervical nerves and/or auricular nerves) using invasive or noninvasive systems can cause slow breathing during sleep and achieve anxiolysis for improved restorative sleep and improved mood and energy levels in the morning. Neuromodulation systems targeting craniocervical (and/or auricular or other peripheral) nerves to modulate immune function may use one or more form of neuromodulation from the list including but not limited to: electrical stimulation (including electrical stimulation comprising one or more of: pulsed electrical stimulation, charge-balanced bidirectional (i.e. AC) waveforms, transdermal techniques, and invasive (i.e. nerve cuff) techniques), ultrasound neuromodulation (including pulsed ultrasound neuromodulation, continuous wave ultrasound neuromodulation, ultrasound neuromodulation that causes neuromodulation by non-thermal mechanisms, ultrasound neuromodulation that causes neuromodulation by thermal mechanisms, and focused ultrasound neuromodulation (e.g. delivered by a phased array)), magnetic stimulation (i.e. TMS), optogenetic neuromodulation, RF neuromodulation, thermal neuromodulation, and other forms of neuromodulation.

### Multi-Channel External Ear Stimulating Electrodes

Described herein are methods and devices for modulating auricular nerves. For example, described herein are modulation apparatuses configured to be worn in or around the outer ear or auricle. In one embodiment, one electrode is worn in, on, or otherwise affixed to the ear (pinna, tragus, inner ear, etc.) on one side of the body and a second electrode is similarly worn in, on or otherwise affixed to the ear on the other side of the body.

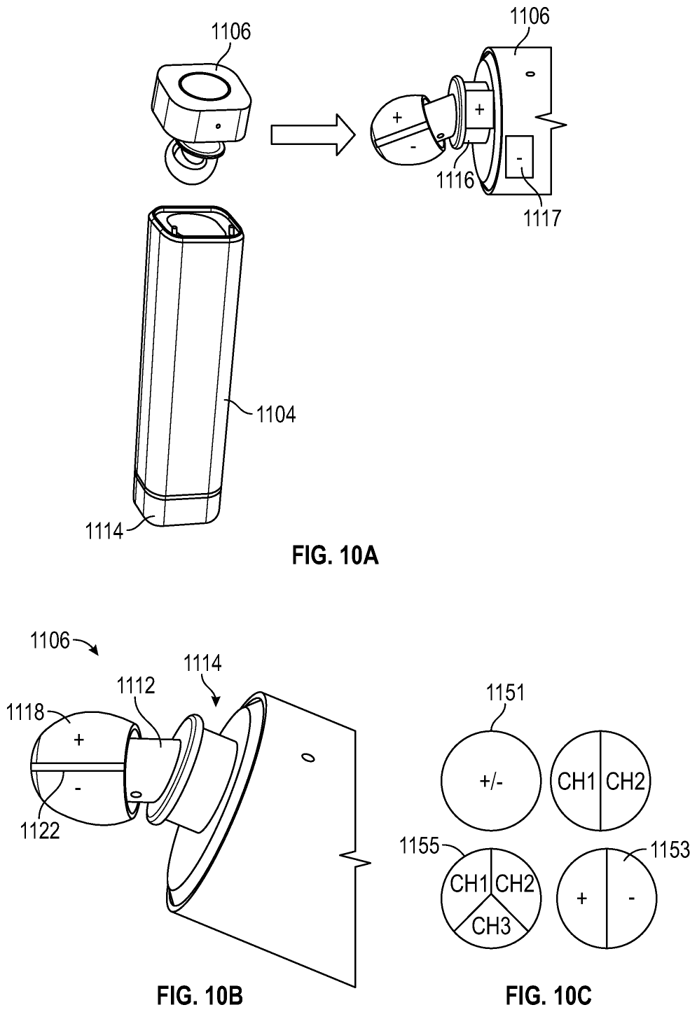

There are several major peripheral nerve branches that innervate the outer ear or auricle or pinna (FIGS. 7A-7C, 8 and 9A-9B). Three are cranial nerves of vagal, facial, and trigeminal origin. Another two branches originate from the C2 and C3 cervical nerves. In addition to a dense supply of peripheral nerve fibers, the skin of the outer ear has a rich network of blood vessels, arteries, and capillaries. In some variations, the methods and apparatuses described herein use biphasic, asymmetric, charge balanced currents where the anodic phase has about four times the current amplitude of the following cathodic phase that is about four times as long as the preceding anodic phase. Methods and devices for delivering rectangular or semi-rectangular, trapezoidal or triangular anodic and cathodic pulses having charge balancing in such a manner can be delivered to auricular nerves, or otherwise target the skin of the external ear in a manner intended to affect physiological processes or biological homeostasis processes including sleep (or wakefulness), appetite (including feeding and satiety behaviors and stimulating or otherwise modifying metabolism), body weight, body temperature regulation, and cognitive processes including attention, working memory, memory encoding, and sleep-dependent memory consolidation.

Other charge balanced ratios of rectangular or other shaped current pulses may be used for example when an anodic phase has about ten percent of the amplitude of the cathodic phase, but is 100 microseconds in duration versus its accompanying shorter cathodic phase that is only 10 microseconds long separated by an interphase gap of greater than 10 microseconds. Other examples may implement combinations of anode and cathode phases that have a duration longer than about 20 or 50 microseconds and an interphase gap longer than about 5 or 10 microseconds in either an asymmetric or symmetrical manner that maintains charge balancing. In embodiments of the described invention pulses may be less than about 500 microseconds or less than 1 millisecond in alternating or pulse current modes. In other embodiments a current offset may be used that produces a change in the electric field of the tissue for a period of time greater than about 10 milliseconds and up to tens of seconds before cycling back through a zero current offset state.

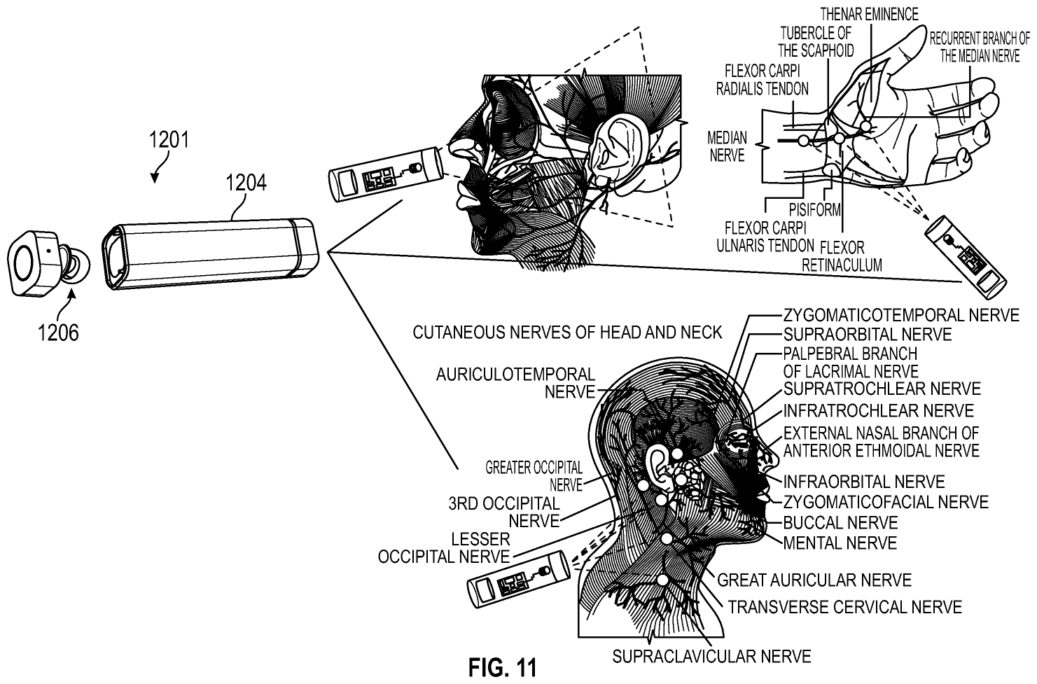

Previous descriptions include the use of TENS methods or tVNS methods that utilize pulse frequencies generally lower than about 150 Hertz, which is not ideally suitable to provide the greatest comfort or effect for a user depending on the desired outcome. To overcome this limitation, any of the methods and apparatuses described herein may include a single or multi-channel external ear stimulating electrode (MEESE) that delivers pulsed electrical stimulation at frequencies greater than about 150 Hertz, like 220, 300, 330, 440, 350, 500, 700, 750, 880, or 900 Hertz for example. These apparatuses and methods may, in some variations, include at least one other channel that transduces visible (light) electromagnetic radiation towards the user's ear, head, or skin (FIGS. 10-12) for either sensing vascular activity or for modulation purposes such as those described in photobiomodulation methods or low-laser therapy methods. Pulsed electrical stimuli having a frequency of about 200 or 300 Hertz or higher may be used to stimulate auricular nerves bias the activity of brain circuits.

The apparatuses described herein may use wireless communication protocols known in the art such as WiFi, WiMax, Bluetooth, cellular protocols, and future methods that may incorporate terahertz waves to provide high speed communication between wearable devices or stationary or portable devices located less than one meter and up to thousands of miles away where at least one of the devices contains one or more neurostimulation channels that is intended to stimulate, modulate, or bias the activity of a cell, nerve, or blood vessel in the external ear using a pulsed electrical current that has a pulse-width of 50 nanoseconds or longer like 1, 10, 20, 50, 100, 300, 500, or 1000 microseconds and an amplitude of 1 nanoampere or higher for example 100, 150, 300, 1000, or 2000 microamperes.

These methods and apparatuses may bias ongoing neurosensory signals in the brain and may include devices that deliver pulsed electrical currents to the skin of the outer ear through one or more channels to discrete regions of the auricle. The device may comprise a system for generating artificial or digitized somatosensory signals at the skin of the auricle or outer ear including the external auditory meatus, concha, tragus, and other fleshy parts of the ear protruding outside the head. The artificial or digitized sensory are defined as being extra-sensory in nature since they are not responding to conventional environmental cues, such as touch, mechanical pressure, temperature, or pain. These artificial, digitized or extrasensory stimulation programs may consist of ultra-low to very high frequency events originating at the superficial skin of the human outer ear to bias brainstem activity and ascending network activity in auditory cortex, prefrontal cortex, somatosensory cortex, visual cortex, limbic centers, motor cortex, or the thalamus as determined through behavioral, electrophysiological, or more salient responses. Another embodiment includes a method of the same for effecting baroreception by way of altering the activity of baroreceptors located in or around the ear achieved by transmitting pulsed electrical currents through the superficial layers of skin on the outer ear.

Also described herein are methods for modulating brainstem activity by delivering ultra-low to high frequency pulsed electrical currents to the skin of the outer ear or pinna (optionally) including the concha, external auditory meatus, tragus, and other anatomical regions of the ear in order to modulate the physiological response characteristics or activity of Merkel's disks, Meissner's corpuscles, Ruffini's corpuscles, and Pacinian corpuscles, as well as other receptor bodies known in the art to be located in the skin.

Also described herein are methods for modulating brainstem activity by delivering ultra-low to very high frequency pulsed electrical currents to the hair follicles of the pinna or outer ear may include the fine hairs in the epidermal layers of the skin lining the external auditory meatus.

Described herein are methods for modulating brainstem activity by delivering ultra-low to high frequency pulsed electrical currents to the skin of the outer ear or pinna (optionally including the concha, external auditory meatus, tragus, and other anatomical regions of the ear) in order to modulate the physiological response characteristics or activity of Merkel's disks, Meissner's corpuscles, Ruffini's corpuscles, and Pacinian corpuscles, as well as other receptor bodies known in the art to be located in the skin.

In some variations described herein, the tissue of the outer ear may be heated less than three degrees Celsius to modulate ongoing neuronal activity in the nerves innervating the external ear, as well as blood flow or enzymatic activity of tissues of the auricle.

For example described herein are devices and methods for delivering digitized signals to the skin of the outer ear for the purposes of enhancing human-machine interactions by affecting somatosensory receptors or neurovascular activity of the outer ear or cochlear blood flow. These methods and apparatuses may regulate cardiovascular function for therapies, diagnostics, or other purposes.

In some variations, the apparatus may include one to twenty independent electrode channels operating in a synchronized, asynchronous, or randomized manner to deliver ultra-low to very high frequency pulsed electrical currents to the outer ear. The multiple channel approaches described herein have been designed to transduce broadband somatosensory stimuli to the skin and nerves of the outer ear. In some variations, the methods for neuromodulation described herein may use tremendously high bandwidth (e.g., spanning about 100 hertz to about ten kilohertz or 50 kilohertz or one or ten megahertz).

There are several major peripheral nerves, which innervate the outer ear and to affect broadband somatosensory stimulus from a physiological perspective the use of multiple, independent channels is preferred.

In some variations the apparatuses described herein are configured as multi-channel external ear neurostimulation electrodes, devices, or systems that may incorporate wireless communication modules known in the art, such as Wi-Fi, Bluetooth, Wi-Max. A MEESE may include terahertz resonators and antennas for communicating with other external devices at high speeds located less than one meter from a neurostimulation device. Another method of the same is described which provides for wireless communication with devices more than one meter away and may include the use of electromagnetic radiation at frequencies in the megahertz, gigahertz, or terahertz range.

An example embodiment comprises a two-pole electrical stimulation circuit wherein one pole is placed in, on, or otherwise affixed to the ear (inner ear, pinna, etc.) on one side of the body—and the other pole is placed on the ear on the other side of the body.

Some variations of the apparatuses include four-channel electrodes that are custom molded for to fit in a user's outer ear. The multi-channel (e.g., three channels, four channels, etc.) electrodes may provide discrete pulsed waveforms to multiple (e.g., three, four, etc.) different regions of the ear that are each mainly innervated by different nerve fibers as described below. The timing of stimuli may be adjusted by a user or control operator, such that alternating regions of the outer ear are stimulated in a sequential manner that repeats for a period of time desired by the user or operator. Neurostimulation adjustments may be made using a user control interface over a network or local device control interface until the desired effects are achieved. Such desired effects may include a change in heart rate, a change in the power of the alpha, beta, or gamma band of EEG signals, a change in perceptual awareness, a change in local blood flow, an amplification, filtering, or demodulation of other sensory (auditory, proprioceptive/postural/balance or visual cues for example) signals processed in the brain stem and cortex. Desired effects may be transient lasting tens to hundreds of milliseconds or chronic lasting hours to days and weeks.

Molds may be constructed of polymers, elastomers, or other plastics and moldable materials known in the art that have electrically conductive properties (and/or in some regions, electrically insulating properties). The process for creating external ear molds that are to be worn in the ear may be conducted on an individual basis with the end user's jaw open when constructing ear molds to ensure a proper fit. Some variations may include an ear mold that is constructed for a normalized ear or for normalized ears of different shapes and sizes for universal fitting rather than personalized molding.

Also described herein are single-channel or multi-channel (e.g., two-channel, four-channel, etc.) outer ear neurostimulators that includes an auditory speaker for delivering concurrent or sequential auditory and somatosensory stimuli. A multichannel stimulator may deliver a neuromodulation sequence, where differential current intensities are delivered to nerves of the outer ear. For example, a maximum power level ratio (total power/neurostim current intensity to individual channel power/current intensity) of 1 may be distributed across at power ratio of 0.4 to the auricular branch of the vagus and approximately 0.2 each to the trigeminal, facial, and cervical nerve branches innervating the skin of the outer ear. Different power rations across various nerves may be used in ranges for each nerve ranging from 0.01 to 0.99 such that the total power spectrum of the neurostimulation programs are dispersed across one or more nerves or anatomical regions of the outer ear.

In some variations the multichannel stimulator delivers a neuromodulation sequence, where the spectral power of neurostimulation frequencies is differentially distributed across nerves of the outer ear. For example, seventy percent of the power in the frequency band ranging from 500 hertz to 1,000 hertz may be delivered to regions of the outer ear chiefly innervated by the auricular branch of the vagus nerve while thirty percent of that power band is directed to the skin of the outer ear that is primarily innervated by the trigeminal nerve. In a similar fashion 95 percent of the power in the neurostimulation frequency band ranging from 2,000 hertz to 2,000,000 hertz may be simultaneously delivered to the trigeminal nerve and five percent of that power may be delivered to the auricular branch of the vagus nerve. Different frequency components of a composite broadband sensory stimulus may be used in ranges from about 200 Hertz to above one kilohertz or above up to, in some cases, about 100 kilohertz such that each nerve or region receives powers of a frequency band ranging from 0.01 to 0.99 such that the total power spectrum of the neurostimulation programs are dispersed across one or more nerves or anatomical regions of the outer ear whereby one region receives stimulation at frequencies less than about one kilohertz and other channels may receive stimulation at frequencies higher than about one kilohertz.

A multichannel external ear neurostimulation device incorporating one or more ultrasound transducers for imaging alterations to hemodynamic activity, blood flow (for example, of arteries in the ear or brain produced by neuromodulation) may be achieved through the delivery of ultra-low to very high frequency pulsed electrical currents to regions of the outer ear inclusive or not of the external auditory meatus. In some variations the apparatuses described herein may be a four-channel electrode composed of conductive silicon, conductive rubber, graphene, or other electroconductive polymer or biocompatible electrically conductive material known in the art may be used to transmit currents to the skin of the outer ear. An embodiment of the electrodes may also include the delivery of low (or ultra-low) to high frequency pulsed electrical currents to the skin lining the ear canal. The electrode should have interleaved nonconducting regions or Non-conducting Insulating Layers (NILs) to enable electrical isolation of channels for independent stimulation of different anatomical regions of the outer ear. Electrodes may have a multi-layer construction composed of a nano-carbon ink printed materials, layers of conductive polymers, or layers of conductive metal fabrics or materials including hydrogel substrates that may be used to improve electrical coupling of a MEESE or similar device to the external ear.

A method for introducing pulsed electrical currents to different locations of the skin's surface from multiple channels in an intelligently scrambled sequence is described. The described architecture provides for complex human machine interactions by enabling uniquely scrambled electrostimulation sequences delivered to discrete body locations in a manner that is tied to environmental, computational, logic, or physiological triggers. Electrical stimulation used for neuromodulation is not restricted to the external ear and similar effects may be achieved using on or more peripheral nerve stimulation sites operating together or in isolation. An embodiment of such may include a wristband to stimulate the median nerve or an ankle band to stimulate nerves innervating the calf and foot. These devices may work in cooperation with external auditory electrodes, but may not need to in order to modulate composite sensory processes like hearing.

As neuromodulation systems become integrated with sensors, sensor bodies or sensor networks there is a need to achieve multiplexed neuromodulation such that neurostimulation can be achieved by delivering pulsed currents to different regions of the body. For example, pulsed stimulation may need to be delivered to the hand or wrist in one set of human machine interactions whereas the skin of an ear may need to be stimulated under another set of human machine interactions.

Intelligent Scrambling of a Neuromodulation Sequence refers to the dynamic nature and requirement for timing commands and cued events that occur across variable sensor network activity. For example, consider a situation where stimulation of nerve afferents of the external ear occur to orient a user's perceptual awareness of a target condition 500 milliseconds after a predetermined digital event occurs. The occurrence of such a digital event, for example the emergence of an object in a digital frame, which repeats at random time intervals several times per hour or more. In addition to this series of neurostimulation events, pulsed electrical signals are commanded to stimulate the peripheral nerves of the wrist or external ear within 50 milliseconds of an analog signal elsewhere in an environmental network (for example, an internet of things or connected home) crossing some predetermined threshold. Thus, any combination of digital and analog cues may trigger events in a manner such that their delivery with respect to one another is Scrambled, but scrambled in an intelligently directed fashion to alter perceptual awareness or to bias sensorimotor networks of the brain including movements and communication.

Another embodiment may include a Scrambled Sensory Neuromodulation process using a multi-channel external ear stimulating electrode (MEESE) previously described. In such an embodiment different channels of the MEESE can be programed to deliver neurostimulation protocols in a customized manner. An exemplary two channel MEESE is described to differentially regulate cardiovascular dynamics and perceptual arousal or awareness. In this example channel 1 of the MEESE provides pulsed electrical signals having a frequency of about 500 hertz to the skin of the right, left, or both external auditory meatus regions of the ear(s) to target predominantly branches of the auricular branch of the vagus nerve. Channel 2 of the MEESE in a similar unilateral or bilateral fashion transmits pulsed electrical currents at a frequency of about 1,000 hertz to the concha. Pulsed electrical stimuli from channel 1 will be triggered for delivery to the external meatus if a heart rate signal crosses some threshold such that heart rate transiently decreases. Pulsed electrical stimuli from channel 2 will be triggered for delivery to the concha to increase perceptual awareness or arousal in reference to an environmental trigger or cue.

An embodiment of the present invention includes a transdermal neurostimulation electrode having a cylindrical, elliptical or irregularly shaped contour with a major axis or diameter less than about 35 millimeters for example 8, 10, 12, 18, 25, or 30 millimeters containing a pulse generator and battery housed within an insulated tube or compartment longer than about 8 millimeters for delivering pulsed currents to the skin in a punctate manner across different regions simultaneously or nearly simultaneously through a conductive polymer or that makes contact with the skin's surface, such as the skin of the outer ear or auricle to alter or perturb composite sensory processes like hearing.

An embodiment of the invention may be comprised using an outer cylindrical, square, oval, or other geometrically symmetrically or asymmetrically shaped layer containing aluminum, titanium, Teflon, or other nonconductive materials to provide non-conductive insulating layers (NIL) for improved stimulation targeting. Neuromodulation may be achieved in such a manner using NILs to deliver transdermal pulsed electrical signals across the skin having an amplitude greater than about 100 microamps up to about 100 milliamps for a minimum of 0.01 seconds at a pulse frequency greater than about 100 Hertz for example 300, 350, 500, 1000, or 10,000 Hertz.

An embodiment of the invention may be comprised using a tube or hollowed compartment of any geometrical shape having a diameter or major axis of about 10 millimeter or larger, such as 15, 20, 25, 30, or 50 millimeters comprised of a material or containing aluminum, titanium, Teflon, or other nonconductive material and may include additional layers or plastics or polymers surrounding the tube or hollowed compartment containing a battery or control circuitry for a transdermal neuromodulation device delivering pulsed electrical signals across the skin having an amplitude greater than about 100 microamps up to about 100 milliamps for a minimum of 1 second at a pulse frequency greater than about 100 Hertz for example 300, 350, 500, 1000, or 10,000 Hertz.

A MEESE that is activated locally by a button or remotely via a connected approach known in the art to deliver a metered dose of neurostimulation or neuromodulation lasting 1, 2, 3, 4, 5, 8, 10, 20, 30 seconds or more like 1, 2, 3, 5, 10, or 20 minutes at some maximum current intensity greater than 100 microamps and less than about 100 milliamps to the external ear in one or more locations for the purpose of altering auditory sensations or perceptions.

Pulsed stimuli delivered from the punctate electrical neuromodulation device or MEESE should have a frequency higher than about 300 hertz such as 350, 500, 1000, 2000, or 10000 hertz to minimize the activation of pain and muscle fibers.

An embodiment of the present invention may have a neuromodulation frequency of about 100, 200, 300, 400, 500, 1000, or 10000 pulses per second delivered in a random or sequential fashion containing the delivery of at least four pulses at one or more frequencies greater than about 130 hertz such as 500 or 1000 Hertz.

An embodiment of the invention includes a tubular or irregular shaped neurostimulation device that also contains a blue-tooth low energy module, Wi-Fi module or other communications architecture known in the art for transmitting stimulus duration and intensity to another device or devices including cloud services upon activation or inactivation of neuromodulation or neurostimulation.

An embodiment of the present invention is intended to provide punctate or focalized transdermal neuromodulation or neurostimulation waveforms to the head, face, ear or back of the ear for more than one second to affect physiological processes or biological homeostasis processes including sleep (or wakefulness), appetite (including feeding and satiety behaviors and stimulating or otherwise modifying metabolism), body weight, body temperature regulation, and cognitive processes including attention, working memory, memory encoding, and sleep-dependent memory consolidation.

A non-invasive neural interface as described herein may be capable of thermally, optically, electrically, or ultrasonically altering or perturbing reticular activity system (RAS) network activity by modulating the activity of craniocervical, auricular, and/or sympathetic nerves or modulating the vascular activity or modulating the skin temperature or any combination thereof via the external ear using thermally, optically, electrically, or acoustically coupled methods to provide a chemical-free approach to restoring poor daily function attributable to sleep loss, stress, or attention and mood disorders. These methods and devices may be used for affecting physiological processes or biological homeostasis including sleep (or wakefulness), appetite (including feeding and satiety behaviors and stimulating or otherwise modifying metabolism), body weight, body temperature regulation, and cognitive processes including attention, working memory, memory encoding, and sleep-dependent memory consolidation. Methods described herein include the modulation, increasing, or decreasing temperature of the skin of the external ear using thermoelectric fabrics or inorganic or organic flexible thermoelectric materials to trigger sleep onset or to modulate sleep duration or sleep cycles by producing Peltier heating or cooling in a thermal generator coupled to the skin through a thermally conductive material shaped to fit the external ear including the external auditory meatus.

Other embodiments include the use of infrared light emitting diodes including organic light emitting diodes emitting about or greater than 690 nm light to alter or increase the temperature of the skin of the external ear including the skin of the external auditory meatus by transmitting light from a light emitting diode or producing underlayer optical heating transmitted through an optically transparent or semi-transparent polymer or material coupled to the external ear to trigger sleep onset, or to modulate sleep duration or sleep cycles. A combination of thermoelectric and optical heat producing and cooling embodiments of the same is described. Other embodiments implement high frequency pulsed electrical currents delivered to the surface of the skin or hair follicles in the skin of the external ear including the skin of the external auditory meatus in a manner that alters the temperature of the skin through changes or alterations in the vascular activity of the external ear like to cause vasodilation or vasoconstriction for inducing sleep onset or to modulate sleep duration, sleep wake transitions or sleep cycles.

In one embodiment, an optically-transparent or semi-transparent membrane interfaces with the skin of the external auditory meatus, such as to be incorporated into an earbud style headphone. A cabled assembly may affix the earbud headphone thermal modulation apparatus to a power source and external control device. In other embodiments the device shall be wireless using communication and control protocols known in the art. In one application an electrically coupled ear bud electrodes may deliver low level electrical currents to the skin of the user to produce changes in the thermodynamic activity of the external ear. Each ear would have a different electrode pole and current would be passed into the skin of the external ear to affect vascular activity. The optically transparent or semi-transparent membrane described may also be optimized as a polymeric material with specific electrical or thermal conductivity properties. In other embodiments the mechanical properties of Coupling Membrane shall be optimized for acoustic coupling to the skin as ultrasound may be used to regulate thermal activity as described. Heating or cooling is generated and produced by a Thermal Generator Underlayer, which may or may not cover, wrap, fill, or surround in a piezoelectric, piezoceramic, or other speaker or sound transducer including ultrasonic ones for delivering digitally-filtered or raw amplified audio stimuli patterns or sound.

The thermal generator underlayer may incorporate one or more infrared light emitting diodes or a rigid or flexible thermoelectric material, which produces optical heating or Peltier heating and cooling. In other embodiments as described ultrasound may be delivered to the surface of the skin in a utilizing a Thermal Generator Underlayer comprised of a siliconized ultrasound transducer or transducer array using capacitive micromachines ultrasonic transducers. In this embodiment pulsed or continuous wave ultrasound will be delivered to the skin to heat neural structures including sympathetic nerves, skin, glands, hair follicles, and vasculature less than one degree Celsius for altering sleep transitions, inducing sleep onset, preserving sleep states, or to modulate attention or physiological arousal.

The devices, methods and systems described above thus far are designed for modulating homeostatic behaviors, such as sleep, appetite, and fat metabolism. In this embodiment the device shall be supported or controlled by a remote or on-board operating system to alter the heating or cooling behaviors of the Thermal Generator Underlayer by controlling the delivery of voltage or current to the light emitting diode or diodes or the thermoelectric materials or ultrasonic transducers as described.

The method of triggering sleep onset shall include a change like an increase or decrease in the surface temperature of the skin by about a degree or two Celsius at a time prior to intended sleep onset. In such an embodiment the cycling of temperature of the external ear using methods and devices described may act as a Sleep Pacemaker. The device shall induce thermal fluctuations by operating the Thermal Generator Underlayer in a continuous, intermittent, cycled, or pulsed mode to preserve power and control thermal loads to device electronics, as well as to more precisely control skin, nerve, and vasculature temperature through heating and cooling where the device is thermally or acoustically coupled to the skin in an efficient manner.

In one embodiment for example heating of one degree is produced at the surface of the skin of the external auditory meatus or other parts including the entire external ear occurs for at least two minutes prior to sleep onset. As described the heating of the skin and modulation of vascular or nerve activity of the external ear is produced by delivering a current or voltage to infrared light emitting diodes or a thermoelectric material. The optically transparent or semi-transparent coupling material transmits light or heat to the skin of the external ear for at least two minutes prior to sleep onset in order to trigger the homeostatic behavior of sleep. In a same embodiment Peltier cooling may be produced by a thermoelectric layer to decrease the temperature of the skin of the external ear or external meatus to trigger sleep onset or to modulate sleep wake cycles. The modulation of sleep through the control of temperature or nerve activity or vascular activity is highly personalized. For many factors and intrinsic biological variables will govern the relationship between body thermostats and sleep states. This is not to mention environmental variables such as room temperature. Therefore in device embodiments it shall be advantageous to design the Thermal Generator Underlayer such that it can rapidly heat or cool the skin or nerves as required to cycle users across sleep wake transitions.

In some embodiments the change in heat of the skin of the external ear, or modulation of neural activity, or modulation of vascular activity or any combination thereof is used to modulate different states of wakefulness by inducing sleep at a time selected by a user or controller or triggered by some other control variable, such as a change in altitude, a change in environmental temperature, a change in user stress, a change in environmental noise levels, a change in ambient or artificial environmental light levels, a change in user body temperature, a change in user heart rate, or change in user blood pressure. In such embodiments cooling of the skin may be used in the same manners by altering sleep and wake cycles where a thermoelectric material produces Peltier cooling. By reducing or increasing the skin temperature or the temperature of vascular beds in the external ear or temperature of sympathetic nerves or sensory nerves of the external ear one can achieve the ability to modulate the homeostatic behavior of sleep using methods, devices, and systems described.

In some embodiments the device will integrate an audio speaker that delivers raw or filtered sound to the user in a manner that is synchronous or asynchronous with changes induced in the skin temperature or changes in local sympathetic nerve or sensory nerve activity or vascular activity of the external ear to modulate sleep wake cycles, sleep onset, sleep duration, subjective sleep quality, sleep wake transitions, or attention. Such embodiments may be used to increase sleep or modulate attention or physiological arousal in individuals suffering from sleep disorders such as insomnia or insomnias. Similarly, embodiments incorporating audio or not may act to regulate sleep patterns or sleep cycles of individuals suffering from arousal disorders, such as hyper-arousal or hyper vigilance.

Other embodiments are intended to produce or trigger increased states of wakefulness in individuals with sleep disorders such as narcolepsy, as well as to modulate attention or sleep in subjects or users suffering from various neurological and neuropsychiatric arousal disorders such as attention deficit disorder, attention deficit hyperactivity disorder, post-traumatic stress, depression, obsessive compulsive disorder, traumatic brain injury, seasonal affective disorder, mild cognitive impairment, mild cognitive or physical impairment associated with hearing disorders or otological or vestibular dysfunctions, concussion, Alzheimer's disease, Parkinson's disease, epilepsy, and others with similar autonomic dysfunctions involving malfunctioning sleep homeostasis.

Some embodiments of the methods and devices described may be used to regulate sleep wake cycles for users operating on space missions or voyages where low-gravity or austere environments can perturb sleep homeostasis. Such an embodiment provides a low weight and low power method of regulating sleep cycles in space which necessitates these features due to space travel payload requirements.

Other devices described herein have been designed to suppress and scramble the endogenous activity of RAS circuits including the LC by modulating cervical plexus nerves, cranial nerves, sympathetic nerves, or auricular nerves of the neck. These nerves transmit primary sensory information directly to the TSNC and influence the activity of the LC and ascending RAS as discussed. By modulating the activity in these ascending arousal networks, transdermal and peripheral neuromodulation thermal, electrical, optical, or ultrasonic waveforms can stimulate physiological relaxation, decrease appetite, boost metabolic activity, regulate the activity of metabolic pathways, regulate body temperature, and alter other biologically homeostatic processes such as sleep.

Effects produced by the methods, devices, and systems described herein can be observed as autonomic changes in heart rate, shifts in heart rate variability, and changes in skin temperature like changes in the temperature of the ear, facial, hand, and foot regions. The effects are also accompanied by a slowing in respiration and a general sense of psychological relaxation.

Utilizing pulsed electrical currents delivered to the skin of the neck or external ear to produce changes in neuronal activity or changes in the temperature of the skin of the neck or external ear shall involve the delivery of high frequency currents typically higher than 200 cycles per second such as 300, 400, 500, 550, 700, 1000, 10000, or 50000 cycles per second in a manner to increase comfort and maximize thermal efficiency. Neuromodulation waveforms may include other features whereby the amplitude of currents are modulated in a temporal pattern such as amplitude modulation or where the frequency of stimulus patterns is further modulated through burst or frequency modulation methods known in the art. Currents transmitted to the skin to alter the temperature of the skin of the neck or external ear or to alter the activity of vascular beds in the neck or the external ear or to alter the activity of neural structures typically will not exceed a peak of 35 milliamps and should maintain a current density less than about 5 milliamps per centimeter squared. For example, in some embodiments a current of about one to five milliamps is delivered to the skin of the external meatus to trigger a change in the skin temperature of the external ear for modulating homeostatic thermostatic mechanisms in humans for altering sleep wake transitions. In such embodiments the intended effect is to produce a change in the thermovascular activity of the ear thereby producing changes to the body's natural thermostatic systems.

### EXAMPLES

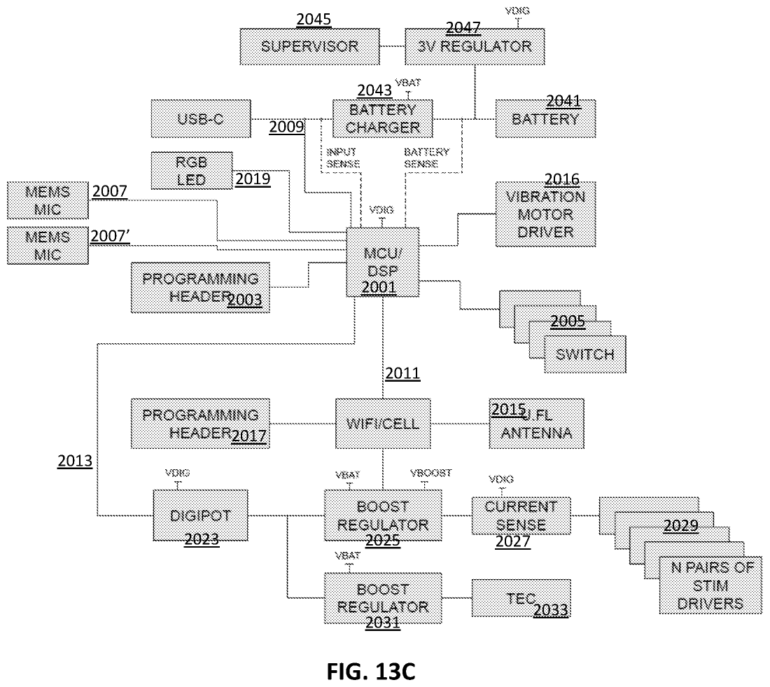

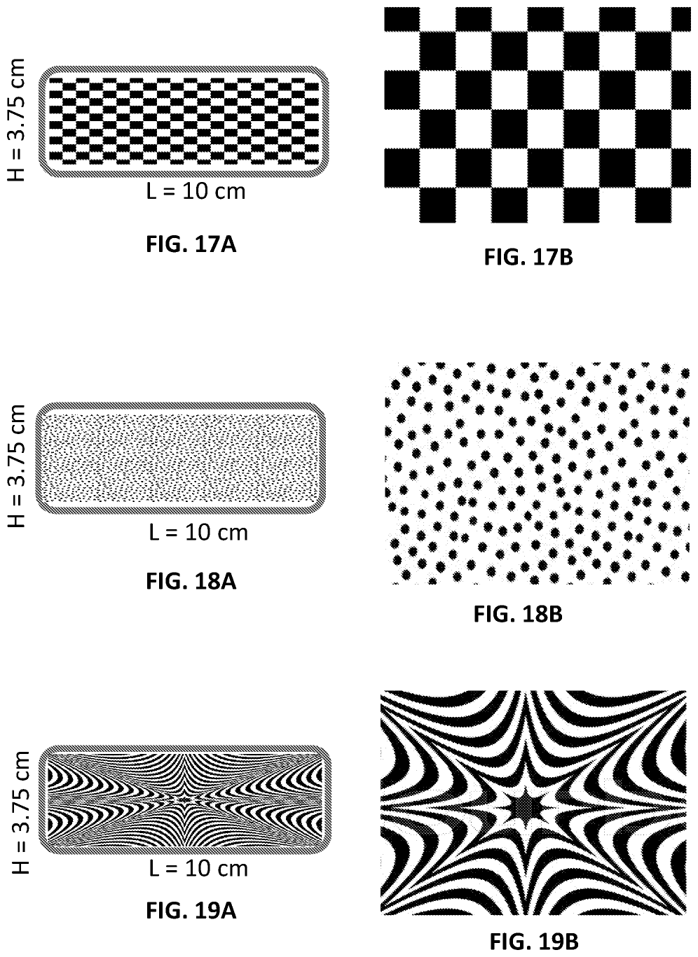

FIG. 13A schematically illustrates one example of an apparatus as described herein. In FIG. 13A, the apparatus includes a pair of earpieces 1911, 1913 each including one or more contacts (e.g., electrical contacts, thermal contacts, etc.). FIG. 13B shows an enlarged view of an earpiece, which includes a low-impedance and compressible hydrogel 1901 on the earpiece, configured to fit into an ear canal so that the hydrogel expands to contact a wall of the ear canal. The hydrogel comprises one or more electrical contacts (e.g., the entire outer region 1905 in FIG. 19). The hydrogel fits (in this example, like a tubular sleeve) over a projection 1919 extending from a base of the earpiece and is in electrical contact with an electrode 1923 on/in the projection. Each earpiece may also include an earpiece housing and a frame or other retention structure 1933 that is configured to secure the earpiece in/on the subject's ear with the hydrogel within the ear canal. In some variations the earpiece does not include an additional retention structure. In some variations the retention structure may include a frame (e.g., a flexible polymeric or metallic frame) that conforms to the outer region of the ear to help hold it in place. In some variations the retention structure may include an adhesive. The retention structure and/or the earpiece housing may include one or more additional contacts (e.g., electrical contacts).

FIG. 13A also shows a neckpiece 1950, configured to be worn or applied to the neck, including the back of the neck, as shown. The neckpiece may include one or more contacts (e.g., electrical, thermal, etc.) for applying a treatment signal to the cervical nerves, as described herein. The neckpiece may be held on manually, or may be worn on the neck. In some variation the neckpiece may be adhesively attached to the neck. In FIG. 13A two earpieces are included; in some variations only a single earpiece is used.

Any of these apparatuses may also include a controller, which may also be coupled with the earpiece(s) and neckpiece and therefore the contacts. The controller may be configured to deliver and coordinate the treatment signals, e.g., electrical and/or thermal and/or mechanical and/or optical, from the contacts in the earpiece(s) and the neckpiece. For example, the controller may coordinate the application of concurrent stimulation treatments from the earpiece(s) and the neckpiece, such as treatment electrical signals comprising a biphasic, pulsed signal having a frequency of greater than 200 Hz to deliver a current density of greater than 2 mA/cm². The apparatus (e.g., system) shown in FIG. 13A also includes wires 1942 coupling the earpieces 1911, 1913 to the controller 1931 on the neckpiece. In this example the controller may be part of the neckpiece; alternatively the controller may be part of the earpiece or may be separate from the earpiece(s) and neckpiece. As mentioned, the earpieces may be configured to secure the apparatus in the subject's ear, so that the hydrogel portion makes contact with the ear canal at one or more locations. The ear pieces may be configured so that the hydrogel may be removed/replaced on the earpiece, e.g., on the protrusion 1919 of the earpiece.

In FIG. 13B, the earpieces each include a body 1914 to which other components attach, such as the projection onto which the hydrogel 1901, 1903 is attached (e.g., formed) onto. The hydrogel 1901, 1903 may be removably attached to the projection 1919. The projection may be a hollow tube or cone-shaped structure. As mentioned, the earpiece neurostimulators 1911, 1913 may also include a retention structure 1933, such as an attachment, for retaining the earpiece in the outer ear (pinna) region. An audio element (not shown) may be included in the apparatus as well.

FIG. 13C schematically illustrate an example of an apparatuses as described herein. In FIG. 13C the apparatus may include a controller 2001 that communicates with: a program header 2003, one or more switches 2005, an inputs (e.g., microphone inputs 2007, 2007′, input ports 2009, wireless/wired inputs 2011, and one or more sensors). Switches may include any appropriate controls (e.g., on/off, increase/decrease signal intensity, etc.). The apparatus may include wires communication and may include one or more antenna 2015, and hardware/software (e.g., programming/programming header(s) 2017). The apparatus may also include one or more outputs (e.g., LEDs 2019, vibration 2016, displays, etc.). In general, the controller may also control output 2013 of the apparatus to apply the treatment signals. This may include controlling electrical treatment signals (via digital controls 2023, boost regulators 2025, current sensing 2027, one or more drivers 2029, etc.). In the variations shown in FIG. 13C, the apparatus is configured to output treatment electrical signals and caloric (e.g., temperature) treatment signals (e.g., treatment thermal signals), via the additional temperature output boost regulator 2031 and heater/cooler (e.g., TEC 2033). In some variations the signal driver 2029 may be connected to an output or jack 2015 that may allow connection to the earpiece and/or neckpiece containing the contacts (e.g., hydrogel contacts). One or more support frames for attaching to the body (e.g., ear, ear canal, neck, etc.) may be coupled to the stimulation outputs and/or inputs, and may house or be connected to the components shown. The contacts may be hydrogel contacts (e.g., low-impedance and compressible hydrogel and/or thermally conductive hydrogel) that, in the earpiece, is configured to fit into a subject's ear canal and expand to contact a wall of the ear canal, not shown in FIG. 13C, but may be included. The apparatus may also include the structures of the neckpiece and/or a hand-held applicator. As mentioned above, the contacts may include a hydrogel that may form one or more contacts (e.g., electrical contacts, thermal contacts, mechanical contacts, optical contacts, etc.).

Any of these systems may also include one or more power regulator(s) including a battery 2041, battery charger 2043, power supervisor 2045 and regulator 2047. Power may be received (charged) via an input such as a USB input 2009, and the controller 2001 may receive data from the charge and/or charging status (battery sense, input sense, etc.).

FIG. 10A illustrates another example of an apparatus that may be used to perform the methods described herein. In this example the apparatus may include a base (storage base 1104) that may house one or more microprocessors, microcontrollers, power banks (e.g., battery), LEDs, USB charging port, USB serial interface, BTLE, WIFI, or other communications hardware and firmware, as well as common environmental sensors, such as a microphone, thermometer, light sensor, accelerometers, GPS, and other sensors/outputs (e.g., displays, etc.). Any of the apparatuses described herein may include one or more of these elements. This base may be optional. The storage base 1104 may store the sensory interface 1106, configured as an earpiece in FIG. 10A. In this example, the earpiece includes one or more outputs (e.g., electrodes, thermal outputs, etc.). In FIG. 10A, the earpiece includes a pair of stimulating electrodes 1108, 1110. In general, these stimulating electrodes may be bipolar or monopolar stimulating electrodes.

The sensory interface (e.g., earpiece) may itself include one or more (e.g., two or more) of following BTLE, WIFI, Battery, Stimulus Amplifiers, Audio Speaker, Piezo element for vibration, Single or Multichannel Controllers LED, Microphone, Accelerometers, EEG sensors and Differential Amplifiers, HR sensors. A second sensory interface, which may be identical of different from the first sensory interface 1106 may also be included. In FIG. 10A, the sensory interfaces are configured to be multi-channel external ear stimulating electrodes (MEESE), as described herein, and may include additional electrical contacts 1116, 1117 for applying sensory biasing signals to the pinna or other body regions.

FIG. 10B shows an enlarged view of one example of a sensory interface 1106 similar to that shown in FIG. 10A, and configured as an earpiece. In this example a projection 1112 extends from an external frame 1114 to be inserted into an ear canal opening. A hydrogel 1118 may be applied over the projection, and may include one or more (in this example, two) contacts, configured in FIG. 10B as electrical contacts. The middle of the hydrogel may be open (e.g., forming a tube-like structure), not shown. The two contact regions may be separated on the post (or in some variations in the hydrogel) by a non-conductive insulating layer (NIL) 1122, thereby dividing the low-impedance and compressible hydrogel into two electrical contacts in this example; the electrical contacts are arranged radially around the projection (and therefore around the hydrogel). As mentioned above, in some variations (as shown in FIG. 10A) the earpiece may also include one or more contacts for other regions of the outer ear (outside of the ear canal), for example by including one or more regions of conductive polymer or other conductive materials to enable the stimulation of different regions of the outer ear if desired through multiple channels.

In variations of these apparatuses that include a base 1104, the base may include one or more additional contacts for applying sensory biasing signals. For example, in FIG. 10A, the bottom of the base is configured to be used as a punctate neurostimulation interface to provide focal stimulation of the skin and nerves for acute treatments (e.g., such as treatments lasting typically less than 1 minute, 2 minutes, 3 minutes, 5 minutes, etc.). The base may contain one or more contacts (e.g., electrical contacts, thermal contacts, etc.) configured as separate channels that may be separated by one or multiple insulating regions. In some variations these contacts may be configured to apply monopolar 1151, bipolar 1153, or tripolar 1155 stimulation in a punctuation manner, e.g., on the subject's skin, when held against the skin. Skin contact may be detected by one or more sensors, including sensors coupled to the electrodes in variations applying electrical stimulation, such as impedance sensors, pressure sensors, etc.

In some variations the base may be configured as a storage and/or charging housing for the one or more earpieces. The base may be configured to store the hydrogel in an airtight and/or humidified chamber to prevent excessive drying (or in some variations to rehydrate) the hydrogel. The base may also be configured to charge the earpiece, particularly in wireless variations. Any of the apparatuses described herein may include a wired connection (e.g., power, control, etc.) between a base and one or more earpieces (not shown).

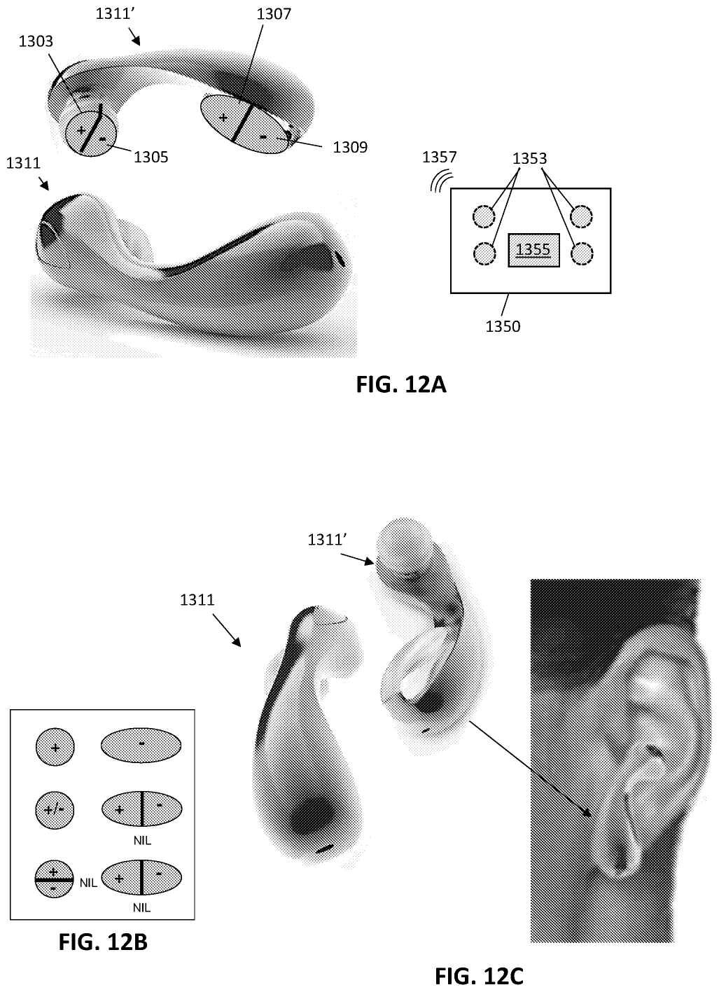

FIG. 11 illustrate operation of an apparatus such as the one shown in FIG. 10A. In this example, the apparatus 1201 includes a storage base 1204 that is a functional punctate neurostimulator that may also apply neurosensory biasing on locations of the head, face, and/or neck, e.g., to modulate composite sensory processes. The system also includes one or more earpieces, configured as shown in FIGS. 10A-10B as a MEESE 1206, to be worn in an ear.

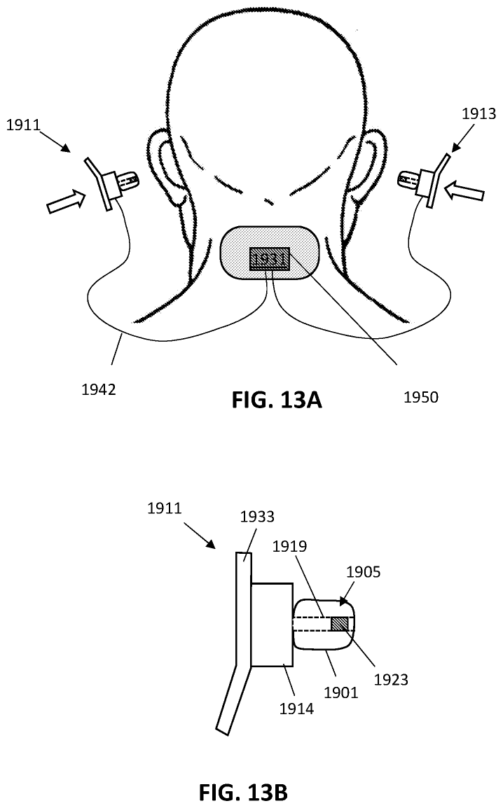

FIGS. 12A-12C illustrates another example of an apparatus (e.g., for delivering scrambled neuromodulation as described herein). In this example, the apparatus includes a pair of ear-worn earpiece devices and a neckpiece 1350 including a plurality of contacts 1353 for delivering treatment stimulation (e.g., thermal, electrical, etc.). The earpiece(s) 1311, 1311′ and neckpiece may be wired or wirelessly 1357 connected to a controller 1355 or may each integrate a controller into the body of one or both earpieces and/or the neckpiece (or in some variations, neckpieces). In the example of FIG. 12A, the earpiece includes one or more electrical contacts 1303, 1305 on protrusion that is configured to extend into the opening of the ear canal. These electrical contacts may be formed of a hydrogel attached to the projection and may be monopolar or bipolar, as shown in FIG. 12C. In this example, the electrodes may be configured as MEESE electrode sites made from conductive polymers or materials in single channel or multichannel bipolar or monopolar configurations. The earpieces may also include one or more (two are shown) electrical contacts 1307, 1309 configured to contact other regions of the ear (as shown in FIG. 12C). In this example, each earpiece 1311, 1311′. As mentioned the body of the earpiece may contain one or more of following BTLE, WIFI, Battery, Stimulus Amplifiers, Audio Speaker, Piezo element for vibration, Single or Multichannel Controllers LED, Microphone, Accelerometers, EEG sensors and Differential Amplifiers, HR sensors.

As mentioned above, any of the apparatuses described herein may include a hydrogel configured to form the sensory biasing signal contact, such as an electrical contact and/or a thermal contact. The hydrogel may be configured to fit into a first ear canal so that the hydrogel expands to contact a wall of the first ear canal. Thus the hydrogel may be compressible. The hydrogel may have a low electrical impedance. For example, in any of these apparatuses, the hydrogel may have an impedance of less than 1.5 KOhms (e.g., less than 1 KOhm, less than 900 Ohms, less than 800 Ohms, between 100-600 Ohms, between 200-500 Ohms, etc.).

The hydrogel may be a silicone hydrogel or a hydrogel including silicone. In some variations the hydrogel may have a water content that is between 40-95% (e.g., between 40-50%, between 40-60%, between 50-60%, between 50-75%, between 60-70%, between 60-85%, between 70-80%, between 70-90%, between 80-95%, etc.). In some variations the hydrogel may be a double network hydrogel.

They hydrogel may incorporate one or more of: and antifungal, an antibacterial, deodorant, etc. For example, the hydrogel may contain an additive such as an antibiotic and/or an anti-fungal, and/or other organic compounds to prevent infection and to control odor. For example, the hydrogel may include an anti-microbial agent, such as one or more of: chlorhexidene acetate, chlorhexideine gluconate, chlorhexidine hydrochloride, and chlorhexidine sulfate, silver acetate, silver benzoate, silvercarbonate, silver iodate, silver iodide, silver lactate, silver laurate, silver nitrate, silver oxide, silver palmitate, silver protein, and silver sulfadiazine, polymyxin, tetracycline, tobramycin, gentamicin, rifampician, bacitracin, neomycin, chloramphenical, oxolinic acid, norfloxacin, nalidix acid, pefloxacin, enoxacin, ciprofloxacin, ampicillin, amoxicillin, piracil, cephalosporins, vancomycin, and bismuth tribromophenate. In some variations, the hydrogel may include an anti-fungal agent such as one or more of: Tolnaftate, Miconazole, Fluconazole, Econazole, Ketoconazole, Itraconazole, Terbinafine, Amphotericin, Nystatin and Natamycin. In some variations, the hydrogel may include an anti-odorant such as one or more of: grapefruit Seed Extract, Tea Tree Oil, Myrtle Oil, and Lemon grass extract.

In any of these apparatuses, the hydrogel may include a color (e.g., dye) to indicate the location of the one or more contacts (e.g., electrical contacts, thermal contacts, etc.). The hydrogel may include a stiffener in order to provide additional strength or support when removing/applying (e.g., replacing) the hydrogel on/off of the earpiece, as described above (see, e.g., FIG. 13A).

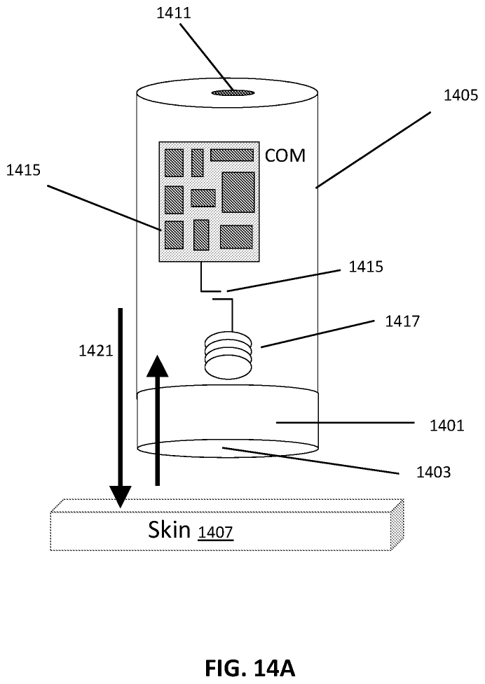

FIGS. 14A-14D illustrate other examples of apparatuses as described herein that may be used for non-invasive auricular and cervical stimulation to modulate multiple integrated neural networks. For example FIG. 14A shows an example of a device for punctate electrical neurosensory biasing configured as an electrical peripheral epidermal neurosensory device (“EPEN”). The device may also include an earpiece (not shown). In FIG. 14A, the apparatus includes a contact 1401 (configured as a button electrode in this example) that includes a conductive polymer or hydrogel for electrically coupling to the skin 1407. The device includes a housing 1405 that may be, for example an insulating tubular housing, which may be composed of, e.g., concentric housing elements or sleeves. The device may include an internal battery or may connect to a separate power source. In FIG. 14A, the device includes a battery connector terminal 1411. A controller (e.g., control circuitry) may be housed within the housing, and may control the application of a treatment signal, such as an electrical treatment signal as described above. The apparatus may also include an internal switch 1415 that may gate the applied signal to the contact 1401. In some variations the contact may be biased (e.g., driven) against the skin by, e.g., a bias, such as a spring or elastomeric material 1417.

The bias may provide a force that engages the switch 1417, so that the neurosensory stimulation is activated only when driven against the tissue (e.g., skin) with sufficient force, as shown by arrows 1421. The skin and underlying tissues (e.g., including muscle and bone) may provide mechanical resistance such that a pushing action of the device against the body activates the neurosensory biasing of neuromodulation circuit; in some variations, deformation forces sufficient to displace the skin by, e.g., about 0.2 mm or more may activate the circuit

The apparatus may be any appropriate size. For example, the apparatus may have a diameter that is less than or about 30 mm, and a length of about 20-70 mm.

FIG. 14B illustrates a similar apparatus for punctate electrical neurosensory biasing having an internal battery 1422, in addition to the charging port 1431 (e.g., microUSB, miniUSB, thunderbolt, etc.). In this example, as in FIG. 14A, the contact 1401 may be configured as an electrical or thermal or mechanical contact. For example the skin-contacting surface 1403 may be a hydrogel as described above and/or a silicone layer. In some variations the contact and skin-contacting surface may be configured for the application of acoustic energy.

In FIG. 14C, the apparatus also includes a rechargeable battery and 1422 and a charging port 1431, but the contact is configured as an ultrasound transducer 1441, and includes a conductive polymer or hydrogel 1403 for making acoustic contact with the skin 1407.

Any of these apparatuses may be configured to telescope in/out. For example, when biasing the contact against the skin, as described above, an inner portion (e.g., inner insulating tube 1447) may be driven into an outer portion (e.g., outer insulating tube 1449), as shown in FIG. 14D.

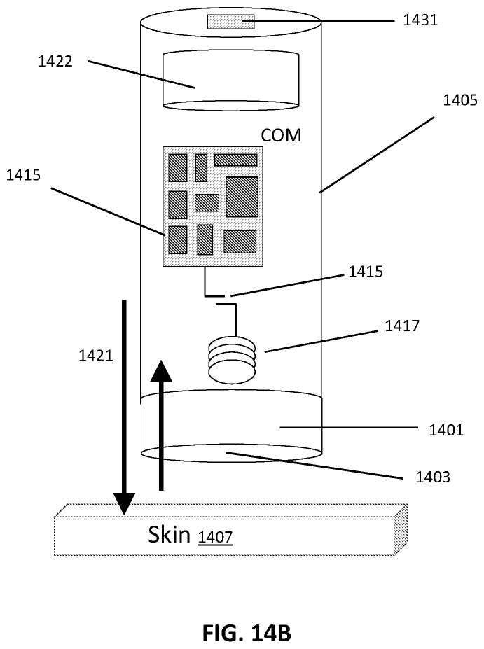

In any of the contacts described herein the applied treatment, such as an applied electrical or thermal treatment, may be distributed over the entire surface of the contact. In particular, when delivering treatment electrical signals it may be particularly beneficial to as uniformly as possible distribute the current to the skin, as the applied current density may be somewhat high. The methods and apparatuses described herein may be configured to uniformly distribute the current even when applying the current through a conductive hydrogel (e.g., a compressed conductive hydrogel). FIG. 15A shows an example of an electrical contact that is configured to deliver punctate electrical neurosensory biasing. In this example, the contact includes multiple layers: an electrical connection (e.g., a magnetic or snap-type electrical connection 1501) at the inner side, then an optional intermediate conductive layer 1503, before a patterned and perforated conductive layer that distributes the current 1505. The patterned and perforated conductive layer may be, for example, a silver-silver chloride (Ag/AgCl) layer that is coated, cut, etched or layered in a manner as shown below to distribute current uniformly across surface of electrode. The perforations may be relatively small. Finally a skin-contacting layer 1507 that may be formed of a conducive polymer or hydrogel may make contact with skin or epidermis in a punctatized manner, and may occupy an area of the skin or epidermis of a minimum of about 0.7 square centimeters up to about 29 square centimeters.

FIG. 15B shows one example of a patterned and perforated conductive layer, showing small (e.g., <0.3 mm diameter, <0.2 mm diameter, <0.1 mm diameter, <0.08 mm diameter, <0.05 mm diameter, etc.) openings. FIG. 15C is another example of a patterned and perforated conductive layer having a striped pattern.

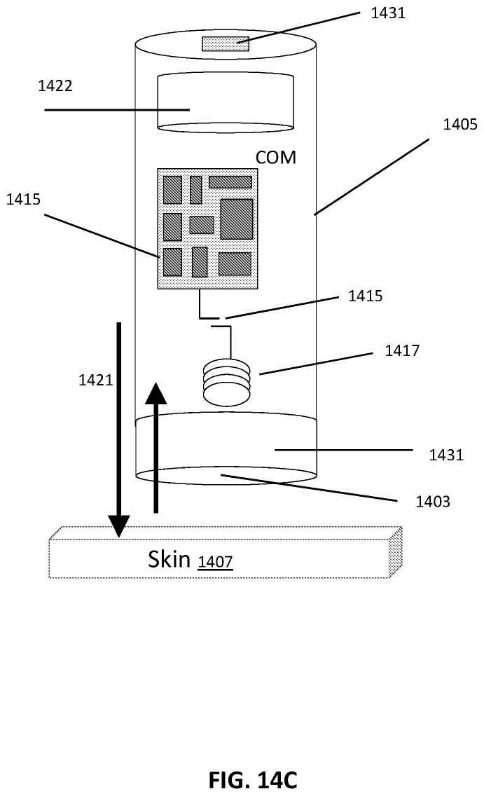

In general, these contacts may be a single uniform contact or they may be divided up into multiple channels, e.g., separated by one or more insulating regions. For example, FIG. 16A shows a single channel contact surface. FIG. 16B is a two-channel (CH1, CH2) surface. FIG. 16C shows a three-channel (CH1, CH2, CH3) surface. Finally FIG. 16 shows a contact surface divided up into two regions (an anode and cathode). Thus, any of these electrical contacts described herein may contain one or more channels to separate by one or multiple insulating regions to achieve monopolar, bipolar, or tripolar stimulation in a punctate manner at the skin's surface. Similarly, thermal contacts may be divided up into different temperature regions (hot/cold, etc.).

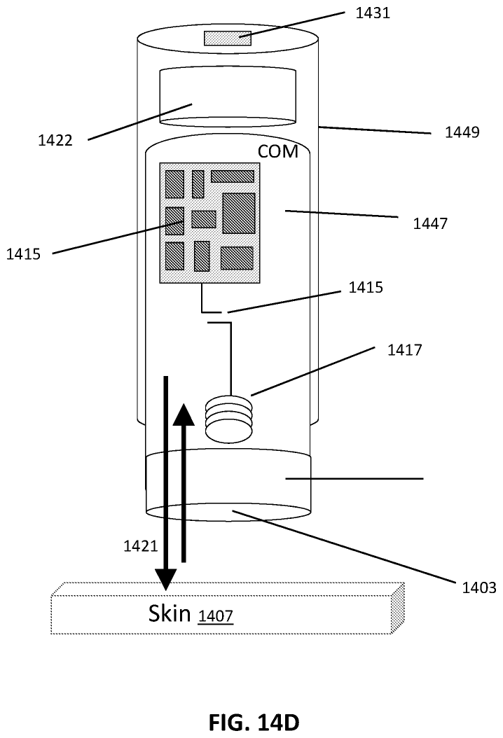

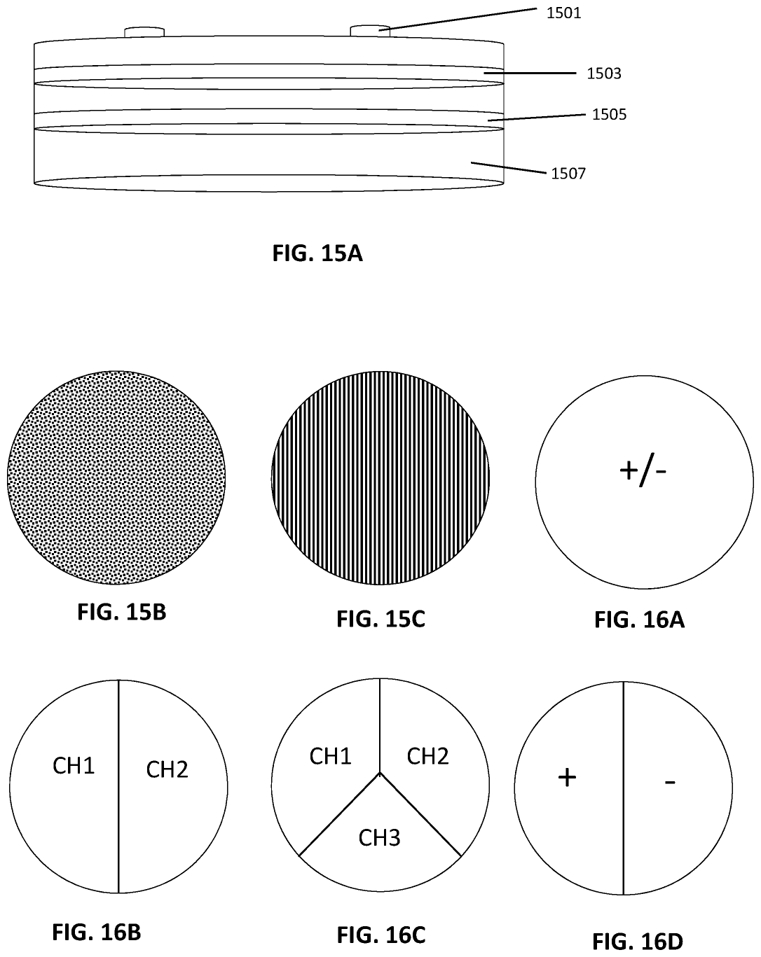

FIGS. 17A-17B, 18A-18B, and 19A-19B illustrate different patterns that may be used for contacts including a patterned and perforated conductive layer. In FIG. 17A an exemplary contact that may be used, e.g., on a neck or other skin region, is shown. This example has a height of 3.75 cm and a length of 10 cm, and the patterned and perforated conductive layer is a checkerboard or grid, show enlarged in FIG. 17B. The grid may be formed of, e.g., an impregnated carbon that may be printed. In FIG. 18A-18B a similar electrode is formed of “nano dots” that may be similarly printed or formed as part of a patterned and perforated conductive layer. As mentioned, this pattern may be formed by inkjet printing. In FIG. 19A-19B, the pattern is shown as a star-shaped pattern (electro-pattern).

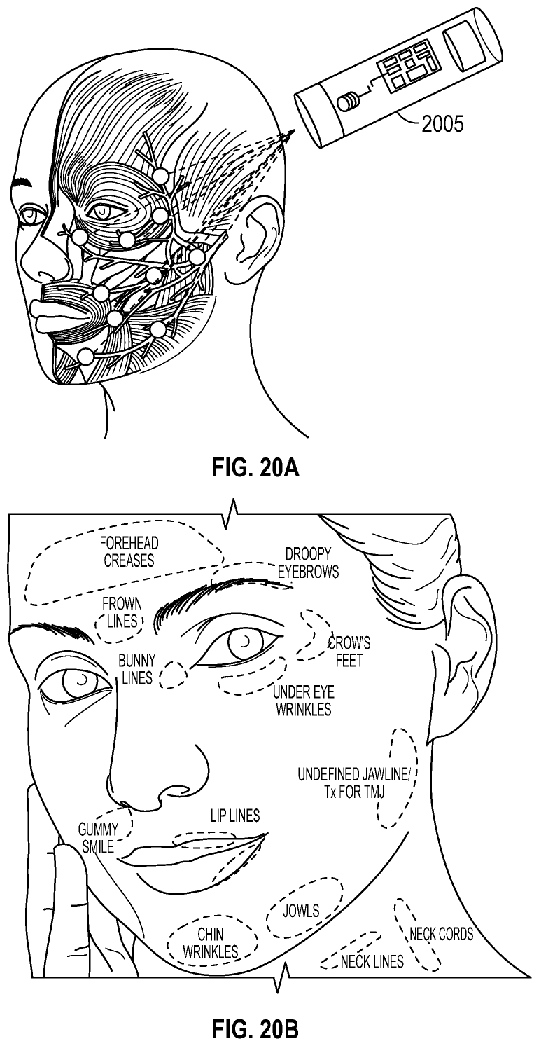

FIGS. 20A-20B, 21, 22 and 23 illustrate alternative indications that may be treated using the apparatuses and methods described herein. For example, in FIG. 20A, an apparatus such as that shown in FIGS. 17A-17D may be used for an aesthetic or cosmetic treatment. FIG. 20B illustrates different treatment zones or region son the face and neck that may be treated either with or without ear (ear canal) stimulation and/or neck (e.g., back of neck) stimulation. For example, an apparatus 2005 (e.g., an EPEN or UPEN neurosensory biasing device) may be used on the facial skin for treatment of wrinkles or to promote facial relaxation or increased blood flow.

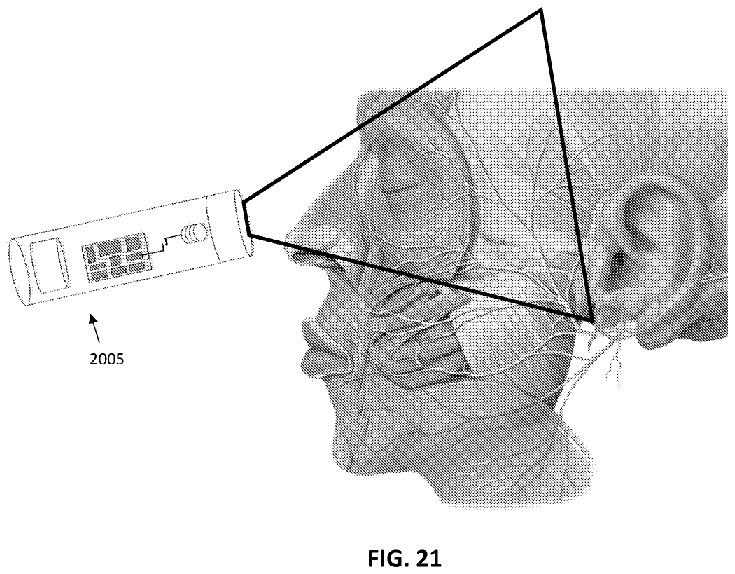

Similarly, FIG. 21 illustrates the use of an apparatus (such as an EPEN or UPEN described above in FIGS. 17A-17D) to apply to a target area for neurosensory biasing of facial and trigeminal afferents to treat Blepharospasm. In this example, the apparatus 2005 may transmit electrical waveforms or ultrasound to the skin and underlying nerves.

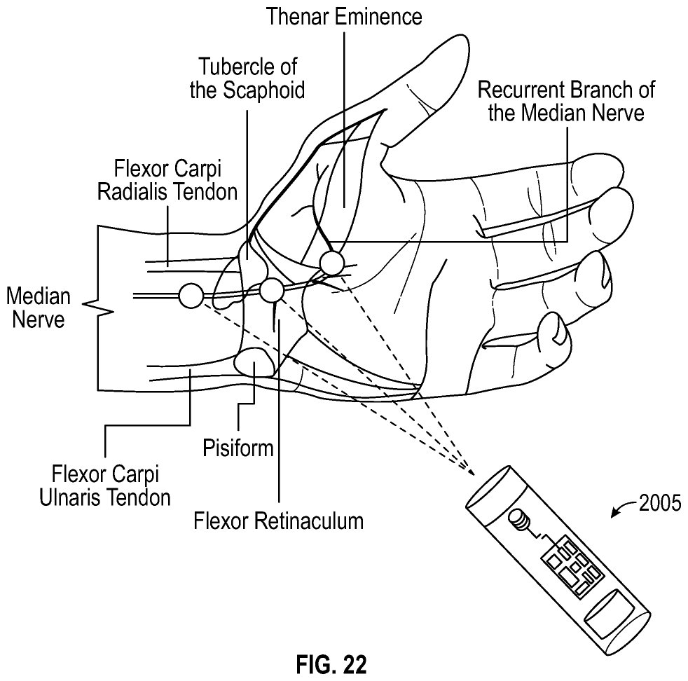

In FIG. 22, the apparatus 2005 (e.g., EPEN or UPEN) may apply neurosensory biasing of median nerve fibers. Target areas may receive neurosensory biasing of median nerve afferents to treat indications such as anxiety, motion sickness, carpal tunnel, or neuropathic pain of the hand or fingers.

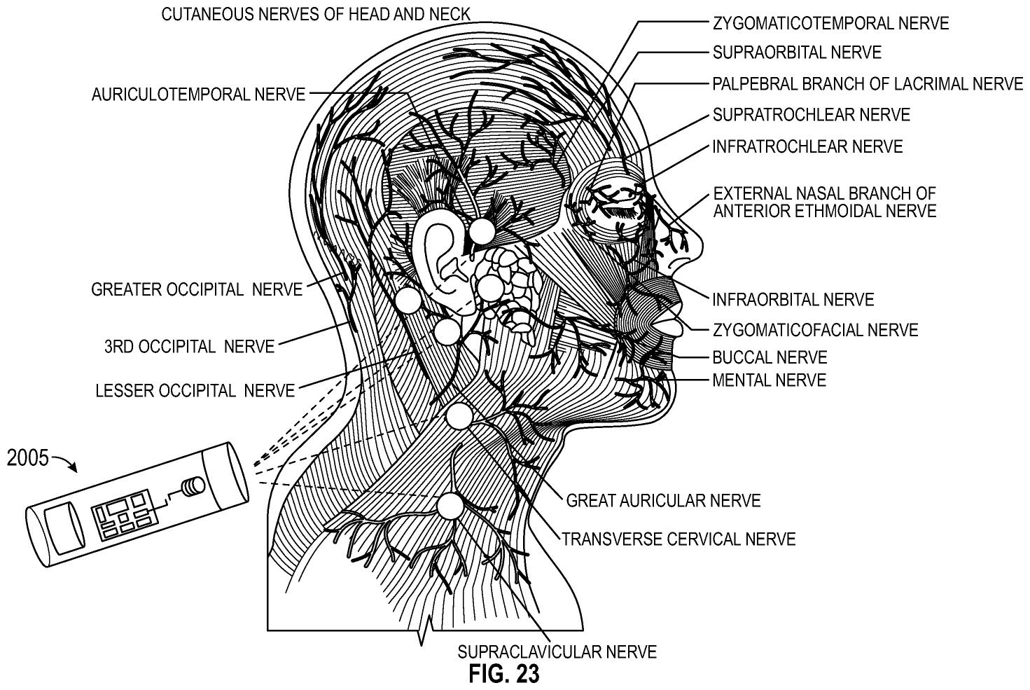

Finally, FIG. 23 illustrates the use of an apparatus 2005 (e.g., an EPEN or UPEN) to apply neurosensory biasing for treatment of motion sickness or nausea to one or more of the areas indicated on the skin, as shown. For example, the apparatus 2005 may target areas for neurosensory biasing of cervical, trigeminal, vagal or other nerve afferents to treat motion sickness or nausea due to organic, environmental, or chemical effects on neural activity.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-108. (canceled)

109. An apparatus, the apparatus comprising:
a first low-impedance and compressible hydrogel configured to fit into a first ear canal so that the hydrogel expands to contact a wall of the first ear canal, wherein the first hydrogel comprises a first electrical contact;
a second low-impedance and compressible hydrogel configured to fit into a second ear canal so that the hydrogel expands to contact a wall of the second ear canal, wherein the second hydrogel comprises a second electrical contact;
a neck contact configured to be worn on the back of a subject's neck, the neck contact comprising one or more neck electrical contacts; and
a controller coupled with the first and second electrical contacts and the one or more neck electrical contacts, the controller configured to deliver a first treatment electrical signal between the first and second electrical contacts and a second treatment electrical signal to the one or more neck contacts, wherein the first treatment electrical signal comprises a biphasic, pulsed signal having a frequency of greater than 200 Hz to deliver a current density of greater than 2 mA/cm².

110. The apparatus of claim 109, wherein the controller is configured to concurrently deliver the first and second treatment electrical signals.

111. The apparatus of claim 109, wherein the second treatment electrical signal is different than the first treatment electrical signal, and comprises a biphasic, pulsed signal having a frequency of greater than 200 Hz.

112. The apparatus of claim 109, wherein the second treatment signal comprises a biphasic, pulsed signal having a frequency of greater than 200 Hz to deliver a current density of greater than 2 mA/cm².

113. The apparatus of claim 109, wherein the first and second hydrogel each have an impedance of less than 1.5 KOhms.

114. The apparatus of claim 109, wherein the first and second hydrogel comprises a silicone hydrogel.

115. The apparatus of claim 109, wherein the controller is configured to deliver the first and second treatment electrical signals for a predetermined time period of between about 5 minutes and 30 minutes.

116. The apparatus of claim 109, wherein the controller is configured as a multimodal controller configured to separately control the application of first and second treatment electrical signals.

117. The apparatus of claim 109, further comprising a speaker configured to emit audio signals into the first ear canal when the first hydrogel is worn in the first ear canal.

118. The apparatus of claim 117, wherein the controller is configured to drive the speaker to emit a tone in conjunction with the treatment electrical signal.

119. The apparatus of claim 109, wherein the controller is configured so that the frequency of the first and/or second treatment electrical signals is greater than 250 Hz.

120. The apparatus of claim 109, wherein the controller is configured so that the current density of the first and/or second treatment electrical signals is 5 mA/cm² or greater.

121. The apparatus of claim 109, wherein the controller is configured so that the first and/or second treatment electrical signals has a pulse width of 150 microseconds or less.

122. The apparatus of claim 109, wherein the first and second hydrogel have a shore A durometer of 70 or less.

123. An apparatus, the apparatus comprising:
a first low-impedance and compressible hydrogel configured to fit into a first ear canal so that the hydrogel expands to contact a wall of the first ear canal, wherein the first hydrogel comprises a first plurality of electrical contacts;
a neck contact configured to be worn on the back of a subject's neck, the neck contact comprising one or more neck electrical contacts; and
a controller coupled with the first plurality of electrical contacts and the one or more neck electrical contact, the controller configured as a multi-channel controller configured to independently deliver treatment electrical signals from one or more of the first plurality of electrical contacts and from the one or more neck electrical contacts, wherein the treatment electrical signals each comprise a biphasic, pulsed signal having a frequency of greater than 200 Hz to deliver a current density of greater than 2 mA/cm².

124. The apparatus of claim 123, further comprising a second low-impedance and compressible hydrogel configured to fit into a second ear canal so that the hydrogel expands to contact a wall of the second ear canal, wherein the second hydrogel comprises a second plurality of electrical contacts.

125. The apparatus of claim 123, wherein the first plurality of electrical contacts are arranged radially around the first ear canal when the device is worn in the first ear canal.

126. The apparatus of claim 123, wherein the first and second hydrogel each have an impedance of less than 1.5 KOhms.

127. The apparatus of claim 123, wherein the first hydrogel comprises a silicone hydrogel.

128. A method of modulating activity of a subject's cranial and cervical nerves and/or auricular nerve, the method comprising:
applying a first treatment signal from a first contact comprising a first hydrogel within a subject's first ear canal, wherein the hydrogel is expanded to contact a wall of the first ear canal; and
applying a second treatment signal from a neck contact on a back of the subject's neck, wherein the first and second treatment signals are concurrently applied, further wherein the first and second treatment signals each comprise either a biphasic, pulsed electrical signal having a frequency of greater than 200 Hz and a current density of >2 mA/cm², or a thermal signal at a frequency of greater than 300 Hz configured to generate a change in temperature of between 0.1 and 3 degrees C.
